(19) United States
(12) Patent Application Publication
(10) Pub. No.: US 2023/0079333 A1
(43) Pub. Date: Mar. 16, 2023

(54) SUPPLEMENTED ANIMAL FEEDS FOR MAMMALS

(71) Applicant: Avivagen Inc., Ottawa (CA)

(72) Inventors: Graham William BURTON, Ottawa (CA); William W. RILEY, Pasig City (PH); James Gary NICKERSON, Charlottetown (CA)

(21) Appl. No.: 17/799,805

(22) PCT Filed: Feb. 15, 2021
(86) PCT No.: PCT/CA2021/050162

§ 371 Date: Aug. 15, 2022

Related U.S. Application Data
(60) Provisional application No. 62/977,990, filed on Feb. 18, 2020.

(51) Int. Cl. A23K 20/179; A23K 50/10; A23K 50/30; A23K 50/40

(52) U.S. Cl. CPC A23K 20/179; A23K 50/10; A23K 50/30; A23K 50/40

(57) ABSTRACT

The present invention features animal feed comprising an effective amount of carotenoid-oxygen copolymer for use in methods for: (i) ameliorating one or more symptoms of subclinical mastitis; (ii) reducing the frequency of subclinical mastitis progressing to full clinical mastitis; (iii) reducing bacteria count in colostrum or milk of a mammal; (iv) reducing physiological stress of a mammal; (v) improving reproductive performance of a mammal; and/or (vi) improving the health of offspring of a mammal. The invention also features a method for producing pasteurized milk from a mammal which has been fed the animal feed supplemented with carotenoid-oxygen copolymer.

### CROSS-REFERENCE TO RELATED APPLICATIONS

The present application claims priority to U.S. Patent Application Ser. No. 62/977,990 filed on Feb. 18, 2020, the content of which is incorporated by reference herein in its entirety.

### BACKGROUND

Mammals raised under modern conditions optimized for growth promotion receive rations containing high proportions of protein, usually in the form of soybean or cottonseed meal, and high percentages of grains such as corn or milo, a type of sorghum. Feed additives have been used to improve the health and well-being of farm animals, including gestating or lactating mammals. Feed is a relatively expensive cost factor in the maintenance of farm animals and production of food from mammals (typically 50 to 70% of the cost). Thus, any improvement in the conversion of feed into food products, including milk, of the mammal, or the enhancement in reproductive performance of the mammal can directly improve the profitability of a food producer.

The use of additives has not been without problems. The widespread use of antibiotics in animal feed promotes the development of antibiotic-resistant microorganisms. As a result of the increasing appearance of antibiotic-resistant bacteria in feed lots and the potential for epidemics caused by antibiotic resistant bacteria, there is increasing governmental pressure to limit the use of antibiotics in animal feed.

Consequently, there is an immediate and increasing need for new, safe, and effective methods for improving the health and well-being of gestating or lactating mammals.

### SUMMARY

The present invention features supplemented animal feeds for use in methods for: (i) ameliorating one or more symptoms of subclinical mastitis; (ii) reducing the frequency of subclinical mastitis progressing to full clinical mastitis; (iii) reducing bacteria count in colostrum or milk of a mammal; (iv) reducing physiological stress of a mammal; (v) improving reproductive performance of a mammal; and/or (vi) improving the health of offspring of a mammal.

In a first aspect, the invention features a method for treating subclinical mastitis in a mammal, the method including feeding the mammal an animal feed including an effective amount of carotenoid-oxygen copolymer. In some embodiments, the mammal is lactating. In further embodiments, the mammal is nursing offspring. The mammal can be cattle, horses, dogs, cats, sheep, or swine. In some embodiments, the mammal is cattle. In particular embodiments, the mammal is dairy cattle. Dairy cattle can be Holstein cattle, Jersey cattle, Brown Swiss cattle, Guernsey cattle, Ayrshire cattle, Milking Shorthorn cattle, or Red and White Holstein. In other embodiments, the mammal is swine. In some embodiments, the feeding is from 10 days to 30 days (e.g., from 10 days to 15 days, from 10 days to 20 days, from 15 days to 20 days, from 15 days to 25 days, from 20 days to 25 days, from 20 days to 30 days, or from 25 days to 30 days) prior to the collection of milk from the mammal. In other embodiments, the feeding is from 30 days to 50 days (e.g., from 30 days to 40 days, from 35 days to 45 days, from 40 days to 50 days, from 30 days to 35 days, from 35 days to 40 days, from 40 days to 45 days, or from 45 days to 50 days) prior to the collection of milk from the mammal. In still other embodiments, the feeding is from 20 days to 45 days (e.g., from 20 days to 30 days, from 25 days to 35 days, from 30 days to 40 days, from 35 days to 45 days, from 21 days to 42 days, from 21 days to 28 days, or from 28 days to 42 days) prior to the collection of milk from the mammal. In some embodiments, the feeding is ongoing during the collection of milk from the mammal. In some embodiments, the shelf-life of the milk is increased. In some embodiments, the method includes ameliorating one or more symptoms of subclinical mastitis. In particular embodiments, the method includes reducing (e.g., by 20%, 30%, 40%, 50%, 60%, 70%, 80%, or more) the frequency of subclinical mastitis progressing to full clinical mastitis in the mammal.

In another aspect, the invention features a method of improving the health of offspring of a mammal, the method including feeding the mammal an animal feed including an effective amount of carotenoid-oxygen copolymer after impregnation of the mammal. The mammal can be cattle, horses, dogs, cats, sheep, or swine. In some embodiments, the mammal is swine. In some embodiments, the feeding is from 10 days to 30 days (e.g., from 10 days to 15 days, from 10 days to 20 days, from 15 days to 20 days, from 15 days to 25 days, from 20 days to 25 days, from 20 days to 30 days, or from 25 days to 30 days) prior to the impregnation of the mammal. In some embodiments, the feeding is ongoing during the gestation period of the offspring. In some embodiments, the method further includes continuing feeding the mammal during the period in which the mammal nurses the offspring. In some embodiments, the transfer of passive immunity from the pregnant mammal to the offspring is enhanced. In particular embodiments, the mammal has or is at risk of subclinical mastitis prior to or subsequent to the impregnation. In particular embodiments, improving the health of offspring includes: (i) reducing the number of stillborn offspring or mummies at birth; (ii) increasing the survival rate of the live offspring from birth to weaning; (iii) increasing weight gain of the offspring from birth to weaning; and/or (iv) reducing the incidence of infectious disease (e.g., diarrhea) in the offspring from birth to weaning. The average increase in survival rate can be greater than 0.5%, preferably greater than 1%, 2%, 3%, 4%, or 5% in comparison to the offspring of the control mammal. The average increase in weight gain in the offspring from birth to weaning can be greater than 0.5%, preferably greater than 1%, 2%, 3%, 4%, or 5% in comparison to the offspring of the control mammal. The average decrease in incidence of infectious disease (e.g., diarrhea) in the offspring from birth to weaning can be greater than 0.5%, preferably greater than 1%, 2%, 3%, 4%, or 5% in comparison to the offspring of the control mammal.

In another aspect, the invention features a method for reducing physiological stress in a lactating mammal, the method including feeding the mammal an animal feed including an effective amount of carotenoid-oxygen copolymer during a period in which the mammal is lactating. In some embodiments, the mammal is nursing offspring. The mammal can be cattle, horses, dogs, cats, sheep, or swine. In some embodiments, the mammal is cattle. In particular embodiments, the mammal is dairy cattle. Dairy cattle can be Holstein cattle, Jersey cattle, Brown Swiss cattle, Guernsey cattle, Ayrshire cattle, Milking Shorthorn cattle, or Red and White Holstein. In other embodiments, the mammal is swine. In some embodiments, reducing physiological stress includes reducing fat loss in the mammal. In particular embodiments, the mammal has or is at risk of subclinical mastitis during the lactation.

In another aspect, the invention features a method for improving reproductive performance of a mammal following the birth of offspring by the mammal, the method including feeding the mammal an animal feed including an effective amount of carotenoid-oxygen copolymer. The mammal can be cattle, horses, dogs, cats, sheep, or swine. In some embodiments, the mammal is cattle. In particular embodiments, the mammal is dairy cattle. Dairy cattle can be Holstein cattle, Jersey cattle, Brown Swiss cattle, Guernsey cattle, Ayrshire cattle, Milking Shorthorn cattle, or Red and White Holstein. In other embodiments, the mammal is swine. In some embodiments, improving reproductive performance is reducing the number of days required for the mammal to return to estrus. In particular embodiments, the mammal has or is at risk of subclinical mastitis prior to or subsequent to impregnation of the mammal.

The invention further features a method for reducing bacteria count in colostrum or milk of a mammal, the method including feeding the mammal an animal feed including an effective amount of carotenoid-oxygen copolymer. In some embodiments, the mammal is lactating. In further embodiments, the mammal is nursing offspring. The mammal can be cattle, horses, dogs, cats, sheep, or swine. In some embodiments, the mammal is cattle. In particular embodiments, the mammal is dairy cattle. Dairy cattle can be Holstein cattle, Jersey cattle, Brown Swiss cattle, Guernsey cattle, Ayrshire cattle, Milking Shorthorn cattle, or Red and White Holstein. In other embodiments, the mammal is swine.

In particular embodiments of the above methods where the mammal is lactating, the method can include feeding the mammal an animal feed including an effective amount of carotenoid-oxygen copolymer, where the feeding is: (i) from 10 days to 30 days (e.g., from 10 days to 15 days, from 10 days to 20 days, from 15 days to 20 days, from 15 days to 25 days, from 20 days to 25 days, from 20 days to 30 days, or from 25 days to 30 days) prior to the collection of milk from the mammal; or (ii) from 30 days to 50 days (e.g., from 30 days to 40 days, from 35 days to 45 days, from 40 days to 50 days, from 30 days to 35 days, from 35 days to 40 days, from 40 days to 45 days, or from 45 days to 50 days) prior to the collection of milk from the mammal; or (iii) from 20 days to 45 days (e.g., from 20 days to 30 days, from 25 days to 35 days, from 30 days to 40 days, from 35 days to 45 days, from 21 days to 42 days, from 21 days to 28 days, or from 28 days to 42 days) prior to the collection of milk from the mammal. In some embodiments, the feeding is ongoing during the collection of milk from the mammal. In some embodiments, the shelf-life of the milk is increased.

In particular embodiments of the above methods where the mammal is impregnated, the method can include feeding the mammal an animal feed including an effective amount of carotenoid-oxygen copolymer, where the feeding is: (i) from 10 days to 30 days (e.g., from 10 days to 15 days, from 10 days to 20 days, from 15 days to 20 days, from 15 days to 25 days, from 20 days to 25 days, from 20 days to 30 days, or from 25 days to 30 days) prior to the impregnation of the mammal; or (ii) from 30 days to 50 days (e.g., from 30 days to 40 days, from 35 days to 45 days, from 40 days to 50 days, from 30 days to 35 days, from 35 days to 40 days, from 40 days to 45 days, or from 45 days to 50 days) prior to the impregnation of the mammal; or (iii) from 20 days to 45 days (e.g., from 20 days to 30 days, from 25 days to 35 days, from 30 days to 40 days, from 35 days to 45 days, from 21 days to 42 days, from 21 days to 28 days, or from 28 days to 42 days) prior to the impregnation of the mammal. In some embodiments, the feeding is ongoing during the entire gestation period of the mammal (e.g., feeding for sows concurrent with a gestation period of about 115 days). In other embodiments, the feeding is ongoing for at least the first half of the gestation period of the mammal. In still other embodiments, the feeding is ongoing for at least the second half of the gestation period of the mammal. In some embodiments, the health of the resulting offspring is improved.

In particular embodiments of any of the above methods, the animal is predominantly (e.g., 80% or more) fed animal feed supplemented with carotenoid-oxygen copolymer. With this approach, the animal feed can include from 0.0001% to 0.005% (w/w) (e.g., from 0.0001% to 0.003% (w/w), from 0.0001% to 0.001% (w/w), from 0.0005% to 0.003% (w/w), from 0.0005% to 0.001% (w/w), from 0.001% to 0.003% (w/w), from 0.001% to 0.005% (w/w), or from 0.003 to 0.005% (w/w)) carotenoid-oxygen copolymer. The animal feed can include from 0.0002% to 0.001% (w/w) carotenoid-oxygen copolymer. In particular embodiments, the animal feed includes from 0.0004% to 0.001% (w/w) carotenoid-oxygen copolymer.

In other embodiments of any of the above methods, the animal is predominantly (e.g., 80% or more) fed animal feed not supplemented with carotenoid-oxygen copolymer but receives at least a single daily feeding supplemented with carotenoid-oxygen copolymer. With this approach, the daily feeding can include a highly supplemented animal feed containing from 0.01% to 0.5% (w/w) (e.g., from 0.01% to 0.1% (w/w), from 0.05% to 0.2% (w/w), from 0.075% to 0.4% (w/w), from 0.15% to 0.4% (w/w), or from 0.2 to 0.5% (w/w)) carotenoid-oxygen copolymer. The animal feed can include from 0.02% to 0.1% (w/w) carotenoid-oxygen copolymer. In particular embodiments, the animal feed includes from 0.05% to 0.1% (w/w) carotenoid-oxygen copolymer. For example, where the mammals are cattle that feed by pasture grazing, the methods of the invention include a daily feeding with the highly supplemented animal feed.

In another aspect, the invention features a method for producing pasteurized milk, the method including: (i) providing milk obtained from a mammal, wherein mammal was fed an animal feed including an effective amount of carotenoid-oxygen copolymer during a period beginning at least from 10 days to 30 days (e.g., from 10 days to 15 days, from 10 days to 20 days, from 15 days to 20 days, from 15 days to 25 days, from 20 days to 25 days, from 20 days to 30 days, or from 25 days to 30 days) prior to the collection of milk from the mammal; and (ii) processing the milk using a low temperature pasteurization process to produce the pasteurized milk. In other embodiments, the mammal was fed an animal feed including an effective amount of carotenoid-oxygen copolymer during a period beginning at least from 30 days to 50 days (e.g., from 30 days to 40 days, from 35 days to 45 days, from 40 days to 50 days, from 30 days to 35 days, from 35 days to 40 days, from 40 days to 45 days, or from 45 days to 50 days) prior to the collection of milk from the mammal. In still other embodiments, the mammal was fed an animal feed including an effective amount of carotenoid-oxygen copolymer during a period beginning at least from 20 days to 45 days (e.g., from 20 days to 30 days, from 25 days to 35 days, from 30 days to 40 days, from 35 days to 45 days, from 21 days to 42 days, from 21 days to 28 days, or from 28 days to 42 days) prior to the collection of milk from the mammal. In some embodiments, the shelf-life of the milk is increased. In other embodiments, the bacteria count in the milk is reduced. The mammal can be cattle, horses, dogs, cats, sheep, or swine. In some embodiments, the mammal is cattle. In particular embodiments, the mammal is dairy cattle. Dairy cattle can be Holstein cattle, Jersey cattle, Brown Swiss cattle, Guernsey cattle, Ayrshire cattle, Milking Shorthorn cattle, or Red and White Holstein.

### Definitions

By “animal” is meant any animal including, without limitation, sheep, swine, cattle, and birds. By “mammal” is meant an animal that has the ability to lactate including, without limitation, sheep, swine, and cattle.

As used herein, “carotenoid” refers to naturally-occurring pigments of the terpenoid group that can be found in plants, algae, bacteria, and certain animals, such as birds and shellfish. Carotenoids include carotenes, which are hydrocarbons (i.e., without oxygen), and their oxygenated derivatives (i.e., xanthophylls). Examples of carotenoids include lycopene; beta-carotene; zeaxanthin; echinenone; isozeaxanthin; astaxanthin; canthaxanthin; lutein; citranaxanthin; beta-apo-8′-carotenic acid ethyl ester; hydroxy carotenoids, such as alloxanthin, apocarotenol, astacene, astaxanthin, capsanthin, capsorubin, carotenediols, carotenetriols, carotenols, cryptoxanthin, decaprenoxanthin, epilutein, fucoxanthin, hydroxycarotenones, hydroxyechinenones, hydroxylycopene, lutein, lycoxanthin, neurosporine, phytoene, phytofluoene, rhodopin, spheroidene, torulene, violaxanthin, and zeaxanthin; and carboxylic carotenoids, such as apocarotenoic acid, 13-apo-8′-carotenoic acid, azafrin, bixin, carboxylcarotenes, crocetin, diapocarotenoic acid, neurosporaxanthin, norbixin, and lycopenoic acid.

As used herein “carotenoid-oxygen copolymer” refers to a carotenoid that has been fully oxidized at its reactive double bonds by spontaneous reaction with molecular oxygen, resulting in copolymers of the carotenoid with oxygen as the main product. In some embodiments, the carotenoid-oxygen copolymer is formed by reacting a carotenoid with up to 6 to 8 molar equivalents of oxygen, or an equivalent amount of oxygen from another oxidizing agent. Such a reaction produces a large proportion of polymeric material (i.e., material having a molecular weight of greater than 1,000 Daltons). The polymeric material is believed to be formed by the many possible chemical combinations of the various oxidized fragments that can be formed from the multiple double bonds. Methods of making carotenoid-oxygen copolymers are described in U.S. Pat. No. 5,475,006 and U.S. Ser. No. 08/527,039, each of which are incorporated herein by reference.

As used herein, “low temperature pasteurization” of milk refers to a method of heating the milk at a temperature ranging from 55° C. to 65° C. (e.g., from 55° C. to 60° C., from 58° C. to 63° C., from 60° C. to 65° C., from 60° C. to 63° C., from 61° C. to 64° C., from 62° C. to 65° C., from 62° C. to 64° C., or from 62° C. to 65° C.) for a time period (e.g., from 10 minutes to 1 hour, from 20 minutes to 40 minutes, from 30 minutes to 1 hour, from 30 minutes to 40 minutes, from 40 minutes to 1 hour, from 1 hour to 2 hours, or from 1 hour to 3 hours) sufficient to reduce the total bacterial count of the milk by 50%, preferably by 55%, 60%, 65%, 70%, 75%, 80%, 85%, 90%, 95%, or 99%.

As used herein, “improving the health of offspring” in a mammal refers to (i) reducing the number of stillborn offspring or mummies at birth; (ii) increasing the survival rate of the live offspring from birth to weaning; (iii) increasing weight gain of the offspring from birth to weaning; and/or (iv) reducing the incidence of infectious disease (e.g., diarrhea) in the offspring from birth to weaning. The improvement is in comparison to control mammals of the same species, age, and condition (e.g., pre-impregnation, gestating, or lactating) that are raised under the same conditions except that the feeds of control mammals are not supplemented with carotenoid-oxygen polymer. The average increase in survival rate can be greater than 0.5%, preferably greater than 1%, 2%, 3%, 4%, or 5% in comparison to the offspring of the control mammal. The average increase in weight gain in the offspring from birth to weaning can be greater than 0.5%, preferably greater than 1%, 2%, 3%, 4%, or 5% in comparison to the offspring of the control mammal. The average decrease in incidence of infectious disease (e.g., diarrhea) in the offspring from birth to weaning can be greater than 0.5%, preferably greater than 1%, 2%, 3%, 4%, or 5% in comparison to the offspring of the control mammal.

As used herein, “feed intake” in a mammal refers to the feed consumed by the mammal daily (e.g., kg/day) per mammal).

As used herein, “reducing physiological stress” in a mammal refers to any one of (i) reducing fat loss in the mammal; (ii) reducing the heart rate; and (iii) reducing the blood pressure. The improvement is in comparison to control mammals of the same species, age, and condition (e.g., pre-impregnation, gestating, or lactating) that are raised under the same conditions except that the feeds of control mammals are not supplemented with carotenoid-oxygen copolymer.

As used herein, “improving reproductive performance” of a mammal refers to (i) the mammal returning to estrus within a shorter period of time; (ii) decreasing the number of undersized offspring at birth; and/or (iii) increasing the number of offspring per litter, in comparison to control mammals of the same species, age, and condition that are raised under the same conditions except that the feeds of control mammals are not supplemented with carotenoid-oxygen copolymer.

As used herein, “OxBC” is the compound containing predominantly carotenoid-oxygen copolymers (e.g., β-carotene-oxygen copolymers), as well as minor amounts of many small molecule oxidation breakdown products, formed by reaction of up to 6 to 8 molar equivalents of oxygen with beta-carotene. In some examples, OxBC is administered as the commercial product, OxC-beta™ Livestock 10% premix.

As used herein, the term “subclinical mastitis” refers to an inflammation of the mammary gland of a mammal caused by a subclinical intramammary infection that does not create visible changes in the milk or the udder. Although the milk appears normal, sub-clinically infected cows generally produce less milk, and the quality of the milk will be reduced.

As used herein, the term “treating subclinical mastitis” refers to (i) ameliorating one or more symptoms of subclinical mastitis or (ii) reducing the frequency of subclinical mastitis progressing to full clinical mastitis. Using the methods of the invention to treat a mammal suffering from subclinical mastitis, the milk production of the mammal can be increased, the quality of the mammal's milk can be improved, and the risk of the subclinical mastitis progressing to full clinical mastitis can be reduced (e.g., by 20%, 30%, 40%, 50%, 60%, 70%, 80%, or more). The effect of (i) ameliorating one or more symptoms of subclinical mastitis or (ii)) reducing the frequency of subclinical mastitis progressing to full clinical mastitis is in comparison to control mammals of the same species, age, and condition (e.g., same severity of subclinical mastitis and status, e.g., pre-impregnation, gestating, and/or lactating) that are raised under the same conditions except that the feeds of control mammals are not supplemented with carotenoid-oxygen copolymer.

Other features and advantages of the invention will be apparent from the following Detailed Description and the claims.

### BRIEF DESCRIPTION OF THE DRAWINGS

FIG. 1 demonstrates the effect of OxBC on dry matter intake (DMI) of the three experimental groups of lactating dairy cows in Example 2: CTR2, T1, and T2.

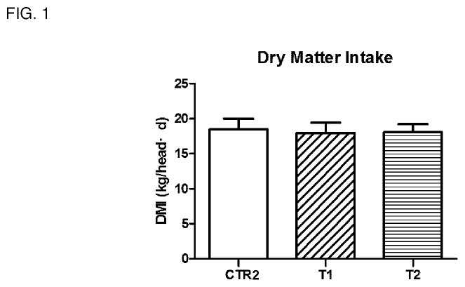

FIG. 2 demonstrates the effect of OxBC on milk production of the three experimental groups of lactating dairy cows in Example 2: CTR2, T1, and T2.

FIG. 3 demonstrates the weather data for the study period. Tₘₐₓ=maximum temperature within a 24-hour period; Tₘᵢₙ=minimum temperature in a 24-hour period; and Precip=total precipitation in a 24 hour period).

FIG. 4 is a violin plot for the natural log of the quarter level SCC for quarters from cows fed OxBC or the control prior to commencement of feeding (D 0) and 21 and 42 days later. In the violin plots the open circle represents the median value, the solid bar the interquartile range, the whiskers the extent of 1.5 times the interquartile range, and the shaded are represents a kernel density plot of the data.

FIG. 5 shows an estimated marginal means for the natural log of the quarter level SCC for quarters from cows fed OxBC (•) or the control (Δ) prior to commencement of feeding (D 0) and 21 and 42 days later.

FIG. 6 shows estimated mean proportion of quarters with an SCC<200,000/mL at Days 21 and 42 after commencement of feeding with OxBC (•) or the control (Δ) diet.

FIG. 7 is a violin plot for the In cow-level (herd test) SCC pre- and post-initiation of feeding of OxBC or of the control diet.

FIG. 8 shows estimated marginal means for the In cow-level (herd test) SCC pre- and post-initiation of feeding of OxBC (•) or of the control (Δ) diet.

FIG. 9 shows estimated marginal mean (95% confidence intervals) daily milk yield (kg/cow/day) at herd tests undertaken prior to commencement of feeding and after the commencement of feeding for cows fed supplementary OxBC (solid orange bars) or fed the control diet (crosshatched blue bars).

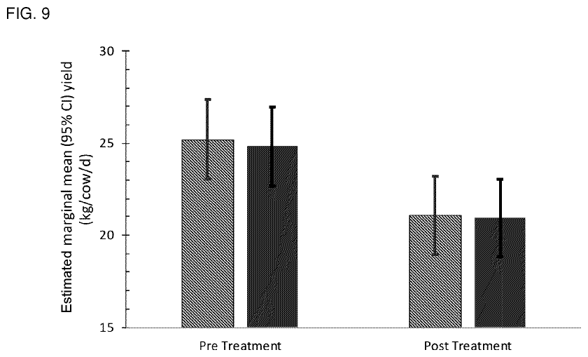

FIG. 10 is a violin plot for the cow-level (herd test) milk fat percentage pre- and post-initiation of feeding of OxBC or of the control diet.

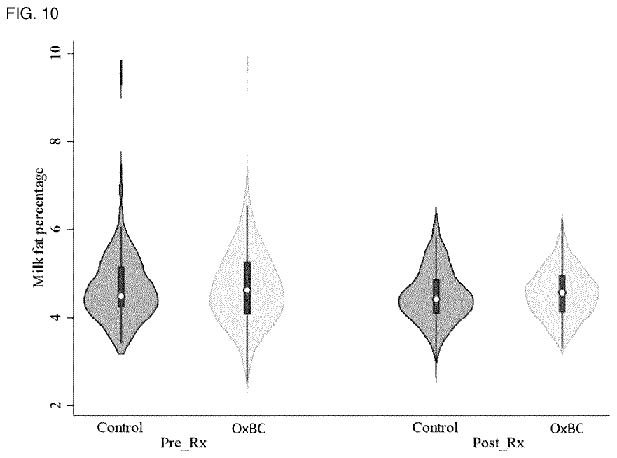

FIG. 11 shows estimated marginal means for the cow-level (herd test) milk fat percentage pre- and post-initiation of feeding of OxBC (•) or of the control (Δ) diet.

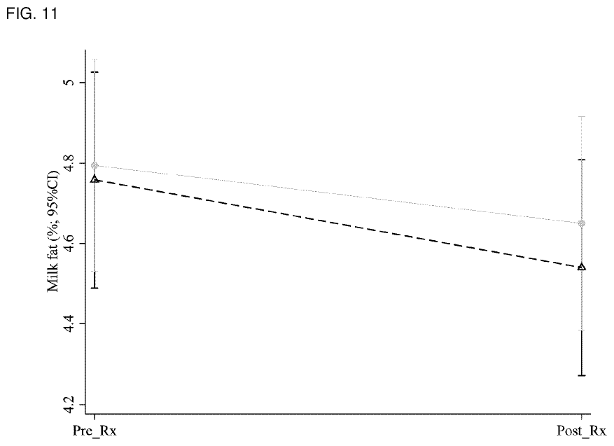

FIG. 12 is a violin plot for the cow-level (herd test) milk protein percentage pre- and post-initiation of feeding of OxBC or of the control diet.

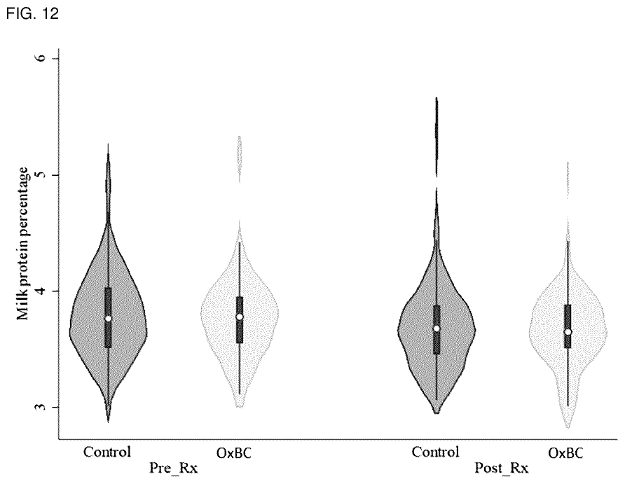

FIG. 13 shows estimated marginal means for the cow-level (herd test) milk protein percentage pre- and post-initiation of feeding of OxBC (•) or of the control (Δ) diet.

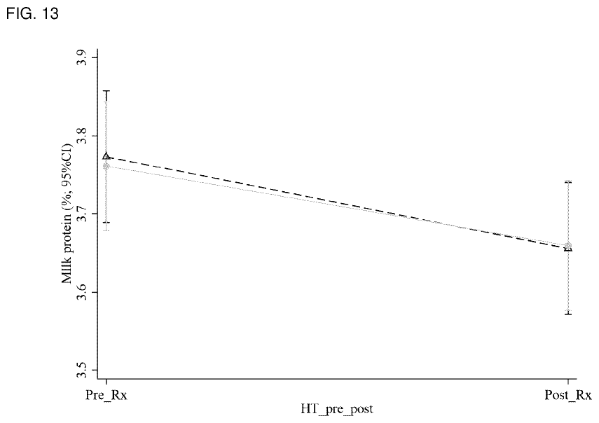

### DETAILED DESCRIPTION

The present invention features methods of treating subclinical mastitis and reducing bacteria count in colostrum or milk of mammals (e.g., cattle, horses, dogs, cats, sheep, or swine) by feeding the mammals an animal feed including carotenoid-oxygen copolymer. The present invention also features methods of reducing physiological stress, improving reproductive performance, and improving offspring health by feeding the mammals an animal feed including carotenoid-oxygen copolymer.

Increased bacteriological cure rate of subclinical intramammary infections (e.g., subclinical mastitis) has been demonstrated following antimicrobial treatment compared with no treatment (see Sol et al., Journal of Dairy Science, 80, 2803-2808 (1997); Oliver et al., Journal of Dairy Science, 87, 2393-2400 (2004); Deluyker et al. Journal of Dairy Science, 88, 604-614 (2005); and Steele and McDougall, New Zealand Veterinary Journal, 62, 38-46 (2014)). However, the economics of treating subclinical cases with antimicrobials with the associated milk discard has been questioned (Swinkels et al., New Zealand Veterinary Journal, 62, 38-46 (2005)), and the potential for increased risk of development of antimicrobial resistance suggests that routine use of antimicrobials for treatment of subclinical infections may not be justified (Barlow, Journal of Mammary Gland Biology and Neoplasia, 16, 385-407 (2009)). Hence alternative, non-antimicrobial approaches would be preferable. The methods of the present invention can provide such a non-antimicrobial approach.

### Administration of Carotenoid-Oxygen Copolymer

Using the methods of the invention, the carotenoid-oxygen copolymer is fed to a mammal in an amount effective for treating subclinical mastitis and/or decreasing bacteria count in the colostrum or milk of the mammal. Using further methods of the invention, the carotenoid-oxygen copolymer is fed to a lactating mammal in an amount effective for reducing physiological stress of a lactating mammal. Using other methods of the invention, the carotenoid-oxygen copolymer is fed to a mammal to improve reproductive performance and increasing the health of offspring of the mammal.

For carotenoid-oxygen copolymer, typical dose ranges are from about 5 μg/kg to about 50 mg/kg of body weight per day. Desirably, a dose of between 5 μg/kg and 5 mg/kg of body weight, or 5 μg/kg and 0.5 mg/kg of body weight per day is fed to the mammal. The exact amount of carotenoid-oxygen copolymer to be administered can depend on such variables as the species, diet, and state of the mammal (e.g., pre-impregnation, gestating, or lactating), and whether the carotenoid-oxygen copolymer is combined with other feed supplements. Standard trials, such as those described in the Examples can be used to optimize the dose and dosing frequency of the carotenoid-oxygen copolymer.

### Animal Feeds

Animal feeds of the present invention can contain carotenoid-oxygen copolymer in an amount effective to reduce bacteria count in the colostrum or milk of mammals or reduce bacteria count in the eggs of birds. Animal feeds of the present invention can contain carotenoid-oxygen copolymer in an amount effective to improve the health of offspring of the mammal and/or improve reproductive performance of mammals. Animal feeds of the present invention can contain carotenoid-oxygen copolymer in an amount effective to reduce physiological stress in lactating mammals.

The animal feeds are generally formulated to provide nutrients in accordance with industry standards. The feeds may be formulated from a variety of different feed ingredients, which are chosen according to market price and availability. Accordingly, some components of the feed may change over time. For discussions on animal feed formulations and NRC guidelines, see Church, Livestock Feeds and Feeding, O&B Books, Inc., Corvallis Oreg. (1984) and Feeds and Nutrition Digest, Ensminger, Oldfield and Heineman eds., Ensminger Publishing Corporation, Clovis, Calif. (1990), each of which is incorporated herein by reference.

Swine and other animal feeds are traditionally balanced based upon protein and energy requirements, and then adjusted if needed to meet other requirements, which will vary for the different stages of growth (e.g., pre-impregnation, gestation, or lactation) and maintenance of the mammal. In some feeding situations, care must be taken to provide the appropriate amino acids as well as overall protein content. For example, swine fed large amounts of corn must have adequate lysine made available in the feed. In most mammal diets, energy requirements are met by starches in cereal grains. Energy requirements may also be met by addition of fat to the feed. Animal feeds containing carotenoid-oxygen copolymer may also be formulated for cattle, horses, dogs, cats, sheep, and birds, among others.

Other ingredients may be added to the animal feed as needed to promote the health and growth of the mammal. The ingredients include, without limitation, sugars, complex carbohydrates, amino acids (e.g., arginine, histidine, isoleucine, leucine, lysine, methionine, phenylalanine, threonine, tryptophan, valine, tyrosine, alanine, aspartic acid, sodium glutamate, glycine, praline, serine, and cysteine, among others), vitamins (e.g., thiamine, riboflavin, pyridoxine, niacin, niacinamide, inositol, choline chloride, calcium pantothenate, biotin, folic acid, ascorbic acid, and vitamins A, B, K, D, E, among others), minerals, protein (e.g., meat meal, fish meal, liquid or powdered egg, fish solubles, whey protein concentrate), oils (e.g., soybean oil), cornstarch, calcium, inorganic phosphate, copper sulfate, and sodium chloride. Any medicament ingredients known in the art may also be added to the animal feed, including, without limitation, antibiotics and hormones. For vitamin, mineral and antibiotic supplementation of animal feeds see Church, Livestock Feeds and Feeding, O&B Books, Inc., Corvallis Oreg. (1984).

Any animal feed blend known in the art can be used in accordance with the present invention, including, without limitation, forages, such as orchard grass, timothy, tall fescue, ryegrass, alfalfa, sainfoin, clovers and vetches, grain feeds, such as corn, wheat, barley sorghum, triticale, rye, canola, and soya beans, crop residues, cereal grains, legume by-products, and other agricultural by-products. In situations where the resulting feed is to be processed or preserved, the feed may be treated with carotenoid-oxygen copolymer before processing or preservation. Desirably, the animal feed of the invention includes rapeseed meal, cottonseed meal, soybean meal, or cornmeal.

Processing may include drying, ensiling, chopping, pelleting, cubing, baling, rolling, tempering, grinding, cracking, popping, extruding, micronizing, roasting, flaking, cooking, and/or exploding. For example, pelleted feed is created by first mixing feed components and then compacting and extruding the feed components through a die with heat and pressure. Animal feeds of the invention can be pelleted as described in, for example, MacBain, Pelleting Animal Feed, American Feed Manufacturers Association, Arlington, Va. (1974), incorporated herein by reference.

Supplemented Diets During Pre-Impregnation, Gestation, and/or Lactation Period

The methods of the invention (e.g., feeding a mammal an animal feed including an effective amount of carotenoid-oxygen copolymer) can be used to improve the health and well-being of gestating or lactating mammals. Advantages of supplementing the diet of an mammal that is about to be impregnated, gestating, and/or lactating include: (i) ameliorating one or more symptoms of subclinical mastitis; (ii) reducing the frequency of subclinical mastitis progressing to full clinical mastitis; (iii) reducing bacteria count in colostrum or milk of the mammal; (iv) reducing physiological stress of the mammal; (v) improving reproductive performance of the mammal; and (vi) improve the health of offspring of the mammal.

### Ameliorating Symptoms of Subclinical Mastitis

The invention features a method of ameliorating one or more symptoms of subclinical mastitis in a lactating mammal where the method includes feeding the mammal an animal feed that includes an effective amount of carotenoid-oxygen copolymer, optionally during a period in which the mammal is lactating. For example, the methods of the invention can be used to treat a mammal suffering from subclinical mastitis, increasing the milk production of the mammal and/or improving the quality of the milk production of the mammal.

### Reducing Subclinical Mastitis Progressing to Full Clinical Mastitis

The invention features a method of reducing the frequency of subclinical mastitis progressing to full clinical mastitis in a lactating mammal where the method includes feeding the mammal an animal feed that includes an effective amount of carotenoid-oxygen copolymer, optionally during a period in which the mammal is lactating. For example, the methods of the invention can be used to treat a mammal suffering from subclinical mastitis, reducing the risk of the subclinical mastitis progressing to full clinical mastitis by 20%, 30%, 40%, 50%, 60%, 70%, 80%, or more.

### Reducing Physiological Stress

The invention features a method of reducing physiological stress in a lactating mammal where the method includes feeding the mammal an animal feed that includes an effective amount of carotenoid-oxygen copolymer during a period in which the mammal is lactating. Lactating causes the mammal physiological stress (e.g., weight loss). Reducing the physiological stress in a lactating mammal increases the health and well-being of the mammal. In an example, the physiological stress of a lactating sow is monitored by the thickness of back fat (e.g., mm of back fat) of the sow.

### Improving Reproductive Performance and Improving the Health of Offspring

The invention features a method of improving reproductive performance of a mammal following the birth of offspring by the mammal where the method includes feeding the mammal an animal feed that includes an effective amount of carotenoid-oxygen copolymer during a period in which the mammal is lactating. Reproductive performance is measured by how quickly the mammal returns to estrus after weaning, the number of undersized offspring at birth, or the number of offspring per litter.

For example, for a sow, improving reproductive performance is indicated by the sow returning to estrus within 7 days (e.g., 6 days, 5 days, 4 days, 3 days, 2 days, or 1 day) of weaning. The reproductive performance is also measured by the number of weak piglets at birth and the total number of piglets per litter. A weak piglet at birth is a piglet that weights<0.7 kg at birth. The reproductive performance is also indicated by the mortality rate of the offspring. An offspring can be live or stillborn.

### Reducing Bacteria Count in Milk

The invention features a method for reducing bacteria count in colostrum or milk of a mammal where the method includes feeding the mammal an animal feed that includes an effective amount of carotenoid-oxygen copolymer. Lowering the total bacteria count in the milk can increase the shelf-life of the milk.

### Supplemented Diets for Pasteurized Milk

The invention further features a method for producing pasteurized milk from a mammal where the mammal was fed an animal feed including an effective amount of carotenoid-oxygen copolymer and processing the milk using a low temperature pasteurization process.

Milk is an excellent medium for bacterial growth. Pasteurization of milk typically includes heating the milk at high temperature (e.g., 70° C. to 75° C.) for about 15 seconds (e.g., about 5 seconds, about 10 seconds, about 20 seconds, about 25 seconds, or about 30 seconds). Low temperature pasteurization is a process where the milk is heated at low temperature (e.g., 55° C. to 65° C.). In low temperature pasteurization, the milk is heated for about 20 minutes (e.g., about 10 minutes, about 15 minutes, about 25 minutes, or about 30 minutes).

Pasteurizing milk increases the safety of the milk and increases the shelf-life of the milk compared to raw milk.

### EXAMPLES

The following examples are put forth to provide those of ordinary skill in the art with a description of how compositions and methods described herein may be used, made, and evaluated, and are intended to be purely exemplary of the invention and are not intended to limit the scope of what the inventors regard as their invention.

### Example 1

### Effect of Dietary OxBC During Gestation and Lactation of Multiparous Sows

The following example was conducted to investigate the effects of dietary OxBC supplementation during gestation and lactation of multiparous sows on productivity and immune status.

### Materials

### Gestating or Lactating Sows

At day 85 of gestation, a total of 150 multiparous sows (Landrace×Yorkshire), ranging from 3rd parity to 8th parity, were randomly allotted by parity, historical reproductive performance, and date of predicted parturition into 3 dietary treatments with 50 sows per treatment. Animals in the control group (CTR1) received basal diet with no supplements; animals in the low dose OxBC group (S1) received basal diet supplemented with 4 mg/kg OxBC; and animals in the high dose OxBC group (S2) received basal diet supplemented with 8 mg/kg OxBC. None of the 3 experimental diets were supplemented with medications. The feeding trial was carried out from day 85 of gestation until day 21 of lactation (at weaning).

At 7 days post-weaning, the weight gain and estrus rate of the lactating sows were recorded. The back fat thickness of the sows was measured with a Pig back fat meter (Renco, USA) at day 85 and day 110 of gestation as well as day 21 of lactation. The average daily feed intake of the sows during lactation was also recorded.

### Diet

A basal commercialized corn-soybean meal diet (Table 1) formulated to meet or exceed the NRC (2012) requirements of gestating and lactating sows served as the basal diet (control diet) for treatment group 1. For treatment groups 2 and 3, OxBC was added to the basal diet at 4 or 8 ppm, respectively. The OxBC that was administered through the feed was the commercial premix product, OxC-beta™ Livestock 10%. OxBC was added to the basal diet at the expense of corn. From day 85 to day 112 of gestation, sows were housed in gestation stalls and provided 3.0 kg feed daily. At 2 days pre-partum, sows were transported to farrowing crates and were fed ad libitum until weaning at day 21 of lactation.

TABLE 1
Compositions of Basal Diet

| Ingredient | Content |
|---|---|
| Corn | 58.64% |
| Soybean meal, 43.0% CP | 23.36% |
| Wheat bran, 15.7% CP | 8.00% |
| Fish meal, 62.8% CP | 2.00% |
| Soybean oil | 4.00% |
| Premix* | 4.00% |
| Total | 100.00 |

*Premix provided following per kilogram of diet:
VA (vitamin A) 12000 IU,
VD (vitamin D) 3000 IU,
VE (vitamin E) 90 IU,
VK (vitamin K) 4.0 mg,
VB₁ (vitamin B₁) 3.0 mg,
VB₂ (vitamin B₂) 10 mg,
VB₆ (vitamin B₆) 4 mg,
VB₁₂ (vitamin B₁₂) 40 μg,
nicotinic acid 40 mg,
pantothenic acid 20 mg,
folic acid 4 mg,
biotin 0.45 mg;
Cu (copper sulfate) 125 mg,
Fe (ferrous sulfate) 150 mg,
I (calcium iodate) 0.25 mg,
Zn (zinc sulfate) 100 mg,
Mn (manganese sulfate) 40 mg,
Se (sodium selenite) 0.25 mg.

TABLE 2
Nutrition Content of Basal Diet

| | Calculated |
|---|---|
| Nutrition | Level |
| DE (Digestible Energy) | 3420 kcal/kg |
| CP (Crude Protein) | 17.9% |
| Ca (Calcium) | 1.02% |
| Total Phosphorus | 0.79% |
| Available Phosphorus | 0.55% |
| Salt (sodium chloride) | 0.41% |
| Lys (Lysine) | 0.96% |
| Met (Methionine) + Cys (Cysteine) | 0.57% |
| Thr (Threonine) | 0.66% |
| Trp (Tryptophan) | 0.23% |

### Methods

### Statistical Analysis

For experimental data, except for estrus rate of sows, one-way ANOVA procedure of SPSS 21.0 (SPSS, INC., Chicago, Ill., USA) was used to determine whether significant variation existed among treatments. When overall differences were found, LSD's multiple range test was used to determine the differences between means. The estrus rate of sows was analyzed using chi-square test. Results were expressed as mean and SEM except for the estrus rate of sows as percentage.

Probabilities<0.05 were regarded as significant, and probabilities>0.05 and <0.10 were regarded as tendencies among treatments.

### Sow Productivity—Offspring

The following criteria of the offspring were measured and recorded from parturition (birth) to weaning: the number of births (total, normal live offspring (birth weight greater than 0.7 kg; no birth defects), stillborn, mummies, weak live offspring (birth weight less than 0.7 kg; no birth defects), or live deformed offspring (birth defect(s)). Each individual piglet's birth weight was recorded, and each litter's average birth weight was calculated. The number of piglets weaned, pre-weaning piglet survival, and diarrhea rate of piglets were also recorded.

At weaning (day 21 of lactation), the number of piglets weaned, incidence of diarrhea, and each individual piglet's weight were recorded. Each litter's average weight, the average daily gain of the piglets, and the survival rate of the piglets were also measured.

### Blood Sample

Blood samples were collected from a subset of 10 sows (n=10) from each dietary treatment. Sows were randomly chosen, and blood (10 mL) was collected by ear venipuncture at 85 days post-coitus, and at 0 days, 14 days, and 21 days postpartum. The same subset of sows was bled at each time period.

Sow's blood was processed for leukocyte phagocytic activity at all 4 times points (85 days post-coitus (“gestation day 85”), and 0 days (“lactation day 0”), 14 days (“lactation day 14”), and 21 days (“lactation day 21”) postpartum). The leukocyte phagocytic activities were determined using a flow cytometer using a Phagotest kit and by following the procedure of Leonard et al., Effect of maternal fish oil and seaweed extract supplementation on colostrum and milk composition, humoral immune response, and performance of suckled piglets. Journal of Animal Science, 88, 2988-2997 (2010).

### Colostrum and Milk Sample

Colostrum and milk were collected from the same sows that were selected for blood sample collection. Colostrum was collected from functional glands within 12 hours postpartum, on lactation day 14, and on lactation day 18 after intramuscular injection of 20 IU oxytocin. Approximately 30 mL was collected each time. Colostrum and milk at lactation day 14 were evaluated for somatic cell count, nutritional composition, immunoglobulins levels, and cytokines levels. The somatic cell counts in colostrum and milk were measured using a flow cytometer (Thermo Fisher, USA).

The OxBC content in milk on lactation day 18 was measured via GC-MS. sCD14 (soluble CD14), cytokines (TNFα, IL-8, IL-18), leukotriene-B4, IgM, IgA, and IgG levels in colostrum and milk at lactation day 14 were measured by ELISA.

The nutritional composition, including fat, protein, and lactose, of the colostrum and milk were also determined.

### Results

### Gestating or Lactating Sows

There were no statistically significant differences among treatments for average daily feed intake during lactation, the back fat thickness at gestation day 110 and lactation day 21, and back fat thickness loss during lactation (Table 3). However, there were dose-dependent trends in both ADFI and back fat thickness. No signs of lack of appetite and clinical disease were found in sows during the whole feeding period.

TABLE 3
Effect of dietary OxBC supplementation during late gestation and lactation of
multiparous sows on feed intake during lactation and back fat thickness of the sows

| | CTR1 | S1 | S2 | P-value |
|---|---|---|---|---|
| Total number of sows | 50 | 50 | 50 | |
| Average daily feed intake | 3.98 ± 0.12 | 4.11 ± 0.08 | 4.16 ± 0.07 | 0.234 |
| during lactation, kg/day/sow | | | | |
| Back fat thickness, mm on day | 16.19 ± 0.23 | 16.27 ± 0.35 | 16.44 ± 0.21 | 0.796 |
| 110 of gestation | | | | |
| Back fat thickness, mm on day | 13.86 ± 0.18 | 14.07 ± 0.29 | 14.29 ± 0.19 | 0.389 |
| 21 of lactation | | | | |
| Loss of back fat thickness, mm | 2.33 ± 0.14 | 2.20 ± 0.12 | 2.15 ± 0.12 | 0.599 |
| during lactation | | | | |

### Sow Productivity—Offspring

Births and Estrus Rate

OxBC treatment did not affect numbers of total piglets born, born alive, stillborn and mummies, and deformed per litter. There were trends to fewer weak piglets and increased birth weight per litter and in Groups S1 and S2 the estrus rate trends were 5% and 6% higher, respectively, than the control group CTR1.

TABLE 4
Effect of dietary OxBC supplementation during late
gestation and lactation of multiparous sows on offspring

| | CTR1 | S1 | S2 | P-value |
|---|---|---|---|---|
| Total number of sows | 50 | 50 | 50 | |
| No. of total born per litter | 11.40 ± 0.37 | 11.48 ± 0.31 | 11.25 ± 0.33 | 0.907 |
| No. of born alive per litter | 10.60 ± 0.33 | 10.55 ± 0.28 | 10.58 ± 0.33 | 0.995 |
| No. of weak piglets** per litter | 0.40 ± 0.10 | 0.35 ± 0.08 | 0.27 ± 0.09 | 0.689 |
| No. of stillborn and mummies | 0.67 ± 0.16 | 0.77 ± 0.17 | 0.56 ± 0.14 | 0.630 |
| per litter | | | | |
| No. of deformed piglets per litter | 0.12 ± 0.05 | 0.16 ± 0.07 | 0.17 ± 0.07 | 0.852 |
| Litter birth weight, kg | 16.45 ± 0.46 | 17.10 ± 0.35 | 16.97 ± 0.53 | 0.580 |
| Individual birth weight, kg | 1.47 ± 0.03 | 1.51 ± 0.03 | 1.53 ± 0.03 | 0.421 |
| Estrus rate of sows during | 85.7 | 90.7 | 91.7 | 0.886 |
| 7 days post-weaning (%) | | | | |

**weak piglets refer to those that were <0.70 kg at birth.

Weaning

Table 5 shows dietary OxBC supplementation tended to enhance litter weight and individual piglet weight at weaning, increase pre-weaning survival and decrease diarrhea rate.

TABLE 5
Effect of dietary OxBC supplementation during late gestation
and lactation of multiparous sows on offspring

| | CTR1 | S1 | S2 | P-value |
|---|---|---|---|---|
| Total number of sows | 50 | 50 | 50 | |
| No. of weaned piglets per litter | 10.21 ± 0.17 | 10.27 ± 0.14 | 10.13 ± 0.12 | 0.756 |
| Litter weight at weaning, kg | 51.33 ± 1.46 | 54.47 ± 1.18 | 55.10 ± 1.04 | 0.073 |
| Individual piglet weight at | 5.49 ± 0.10 | 5.72 ± 0.09 | 5.76 ± 0.08 | 0.089 |
| weaning, kg | | | | |
| Average daily gain, | 189.88 ± 4.56 | 197.47 ± 4.54 | 199.73 ± 3.58 | 0.183 |
| g/day/piglet | | | | |
| Pre-weaning survival, % | 91.47 ± 1.21 | 92.94 ± 1.13 | 94.64 ± 1.01 | 0.332 |
| Diarrhea rate of piglets, % | 3.19 ± 0.52 | 2.27 ± 0.46 | 2.01 ± 0.24 | 0.138 |

### Blood Sample

As described in Table 6, the neutrophil phagocytic activity in whole blood of sows were unaffected by dietary OxBC supplementation.

Regardless of dietary treatments, the neutrophil phagocytic activity is highest at gestation day 85, and decreased to the lowest at parturition, and rebounded to higher values at lactation day 14 and lactation day 21.

TABLE 6
Effect of dietary OxBC supplementation during late gestation and
lactation of multiparous sows on neutrophils phagocytic activity
in whole blood the sows

| | CTR1 | S1 | S2 | P-value |
|---|---|---|---|---|
| Total number of sows | 10 | 10 | 10 | |
| Day 85 of gestation | 91.14 ± 1.96 | 90.88 ± 2.86 | 90.35 ± 1.18 | 0.849 |
| Parturition | 70.40 ± 2.97 | 75.08 ± 2.88 | 74.87 ± 2.16 | 0.404 |
| Day 14 of lactation | 80.34 ± 3.43 | 80.67 ± 3.38 | 83.39 ± 3.19 | 0.477 |
| Day 21 of lactation | 84.80 ± 2.74 | 85.41 ± 2.18 | 86.40 ± 1.66 | 0.879 |

### Colostrum and Milk Sample

Somatic Cell Count (SCC)

As summarized in Table 7 and Table 8, although the differences in nutritional composition and somatic cell count (SCC) in colostrum and milk of multiparous sows did not rise to statistical significance, there were tendencies to increased % Fat and decreased SCC. There was a trend to increased lactose concentration in milk at lactation day 14 with increasing dietary OxBC level (P=0.071).

TABLE 7
Effect of dietary OxBC supplementation during late gestation and
lactation of multiparous sows on the nutritional composition
and somatic cell count (SCC) in colostrum of the sows

| | CTR1 | S1 | S2 | P-value |
|---|---|---|---|---|
| Total number of sows | 10 | 10 | 10 | |
| Fat, % | 3.29 ± 0.16 | 3.74 ± 0.28 | 3.48 ± 0.28 | 0.448 |
| Solid-not-fat, % | 20.25 ± 0.65 | 19.81 ± 0.78 | 20.78 ± 0.97 | 0.703 |
| Protein, % | 7.65 ± 0.24 | 7.48 ± 0.31 | 7.86 ± 0.37 | 0.690 |
| Lactose, % | 11.21 ± 0.36 | 10.95 ± 0.45 | 11.51 ± 0.55 | 0.694 |
| SCC, ×10⁶/mL | 5.34 ± 0.37 | 4.93 ± 0.34 | 4.98 ± 0.39 | 0.696 |

TABLE 8
Effect of dietary OxBC supplementation during late gestation and
lactation of multiparous sows on the nutritional composition
and somatic cell count (SCC) in day 14 milk of the sows

| | CTR1 | S1 | S2 | P-value |
|---|---|---|---|---|
| Total number of sows | 10 | 10 | 10 | |
| Fat, % | 4.96 ± 0.40 | 5.64 ± 0.31 | 5.30 ± 0.16 | 0.300 |
| Solid-not-fat, % | 11.40 ± 0.32 | 10.81 ± 0.25 | 11.10 ± 0.23 | 0.195 |
| Protein, % | 3.86 ± 0.10 | 4.02 ± 0.11 | 4.13 ± 0.08 | 0.173 |
| Lactose, % | 5.67 ± 0.13 | 5.93 ± 0.12 | 6.09 ± 0.11 | 0.071 |
| SCC, ×10⁶/mL | 4.03 ± 0.30 | 3.52 ± 0.33 | 3.34 ± 0.32 | 0.300 |

Immunoglobulin Level

Table 9 and Table 10 show the effects of dietary OxBC supplementation during late gestation and lactation on immunoglobulins concentration in colostrum and milk of multiparous sows. Dietary supplementation with OxBC greatly enhanced colostral immunoglobulin levels including IgM, IgA and IgG (P<0.05), as well as IgM (P<0.05) and IgG level in milk (P=0.052) at lactation day 14. As for dose response, group S2 trends had greater IgM, IgA and IgG levels in colostrum at lactation day 14 and greater IgM and IgG level in milk at lactation day 14 than group S1.

TABLE 9
Effect of dietary OxBC supplementation during late gestation and
lactation of multiparous sows on immunoglobulins level in
colostrum at lactation day 14 of the sows

| | CTR1 | S1 | S2 | P-value |
|---|---|---|---|---|
| Total number | 10 | 10 | 10 | |
| of sows | | | | |
| IgM, mg/mL | 2.55 ± 0.35^{b} | 4.38 ± 0.52^{a} | 4.52 ± 0.42^{a} | 0.005 |
| IgA, mg/mL | 2.39 ± 029^{b} | 4.34 ± 0.40^{a} | 5.11 ± 0.52^{a} | 0.001 |
| IgG, mg/mL | 26.91 ± 2.09^{b} | 28.97 ± 1.89^{ab} | 33.22 ± 1.628 | 0.024 |

TABLE 10
Effect of dietary OxBC supplementation during late gestation and
lactation of multiparous sows on immunoglobulins level in
day 14 milk of the sows

| | CTR1 | S1 | S2 | P-value |
|---|---|---|---|---|
| Total number | 10 | 10 | 10 | |
| of sows | | | | |
| IgM, mg/mL | 0.024 ± 0.005^{b} | 0.051 ± 0.010^{a} | 0.057 ± 0.011^{a} | 0.023 |
| IgA, mg/mL | 0.26 ± 0.06 | 0.34 ± 0.07 | 0.29 ± 0.08 | 0.764 |
| IgG, mg/mL | 1.26 ± 0.12 | 1.70 ± 0.29 | 2.02 ± 0.30 | 0.052 |

Cytokine Level

As shown in Table 11 and Table 12, dietary OxBC supplementation during late gestation and lactation decreased TNF-α and IL8 level in colostrum, as well as TNF-α and IL18 level in milk (P<0.05), while it tended to increase sCD14 level in milk (P=0.055) at lactation day 14. As for dose response, compared with sows fed 4 mg/kg OxBC diet, sows fed 8 mg/kg OxBC diet had numerically decreased TNF-α, IL8 level in colostrum, decreased TNF-α level milk, and increased sCD14 level in milk at lactation day 14, but those indexes between the two OxBC treated groups had no statistical differences (P>0.05).

TABLE 11
Effect of dietary OxBC supplementation during late gestation and lactation
of multiparous sows on cytokines level in colostrum of the sows

| | CTR1 | S1 | S2 | P-value |
|---|---|---|---|---|
| Total number of sows | 10 | 10 | 10 | |
| TNF-α, ng/mL | 0.34 ± 0.13^{a} | 0.10 ± 0.03^{b} | 0.08 ± 0.02^{b} | 0.044 |
| IL8, pg/mL | 1079.06 ± 113.80^{a} | 982.27 ± 68.81^{ab} | 605.46 ± 67^{b} | 0.001 |
| IL18, pg/mL | 97.39 ± 14.25 | 109.09 ± 7.08 | 78.52 ± 16.82 | 0.338 |
| sCD14, ng/mL | 15.36 ± 1.35 | 18.90 ± 2.67 | 25.43 ± 9.35 | 0.185 |
| LTB4, ng/mL | 1.40 ± 0.36 | 0.98 ± 0.15 | 1.10 ± 0.35 | 0.498 |

TABLE 12
Effect of dietary OxBC supplementation during late gestation and lactation
of multiparous sows on cytokines level in milk of the sows

| | CTR1 | S1 | S2 | P-value |
|---|---|---|---|---|
| Total number of sows | 10 | 10 | 10 | |
| TNF-α, ng/mL | 0.38 ± 0.16^{a} | 0.19 ± 0.07^{b} | 0.06 ± 0.01^{b} | 0.035 |
| IL8, pg/mL | 986.33 ± 135.88 | 956.04 ± 66.94 | 973.27 ± 136.81 | 0.938 |
| IL18, pg/mL | 315.37 ± 47.32^{a} | 114.93 ± 15.23^{b} | 175.78 ± 41.04^{b} | 0.004 |
| sCD14, ng/mL | 8.04 ± 1.19 | 10.90 ± 1.31 | 13.55 ± 1.84 | 0.055 |
| LTB4, ng/mL | 1.60 ± 0.35 | 1.54 ± 0.47 | 1.33 ± 0.29 | 0.618 |

OxBC Level

The OxBC content was measured via measuring geronic acid content. At lactation day 18, 30 mL milk/sow from 10 sows/treatment were collected. However, the minimum milk requirement for geronic acid measurement is 200 mL. Therefore, identical volumes (20 mL) from the 10 sows were mixed to obtain sufficient volume (200 mL) for a single measurement. The values of geronic acid content in milk for group CTR1, S1, and S2 were 5.99 ng/mL, 7.69 ng/mL, and 11.02 ng/mL, respectively. The corresponding calculated level of OxBC content in the milk for group CTR1, S1, and S2 were 0.30 mg/mL, 0.38 mg/mL, and 0.55 mg/mL, respectively.

### Conclusion

No signs of inappetence and clinical disease were found in sows throughout the feeding period. Dietary supplementation with OxBC during late gestation and lactation enhanced lactation performance of multiparous sows, indicated by increased litter weight and individual piglet weight at weaning. The mechanism behind the improvement may be explained by improved immunoglobin levels and decreased proinflammatory factor levels in colostrum and milk. Thus, the dietary OxBC supplementation during late gestation and lactation shows beneficial effects on immune status of multiparous sows, which will support the health of their nursing offspring.

### Example 2

### Effect of Dietary OxBC on Mastitis Treatment of Lactating Dairy Cows

The following study was conducted to investigate the ability of dietary OxBC supplementation in preventing or reducing mastitis in dairy cattle.

### Materials

Thirty lactating Holstein dairy cows (body weight: 655.3±81.9 kg) with clinical mastitis were selected for the trial. Cows were acclimated for a period of 10 days prior to starting the trial to allow sufficient time to determine baseline somatic cell count (SCC). Cows were equally divided into one of three dietary treatment groups (10 animals per treatment): control, low dose OxBC, and high dose OxBC.

The basal diet for each treatment group was a total mixed ration (TMR). Animals in the control group (CTR2) received basal diet with no supplements or medications; animals in the low dose OxBC group (T1) received basal diet supplemented with 30 ppm OxBC; and animals in the high dose OxBC group (T2) received basal diet supplemented with 60 ppm OxBC. The study period lasted for 45 days, including 10 days of adaptation.

### Methods

Each cow was marked by ear tag for identification. All animals were managed according to the standard operating procedure (SOP) of the farm including vaccinations, health interventions, diet formulations, and source of feed.

The evaluation criteria included the following: dry matter intake (DMI) for each group of animals was determined daily; the total weight (kg) of milk produced by each animal (milk yield) was recorded every 2 days; and the somatic cell count (SCC), total bacterial count (TBC), and the protein, fat, and lactose content of the milk samples collected on a weekly basis from each animal were measured.

Data obtained were subjected to a GLM procedure of SAS (Version 9.4; SAS Institute, USA). A Duncan significant difference test procedure was used to determine the differences among means. Significance was declared at P≤0.05.

### Results

### Dry Matter Intake (DMI)

The DMI of the CTR2 group was 18.48 kg/day, and the DMI of T1 group and T2 group were 17.96 kg/day and 18.07 kg/day, respectively.

### Milk Production

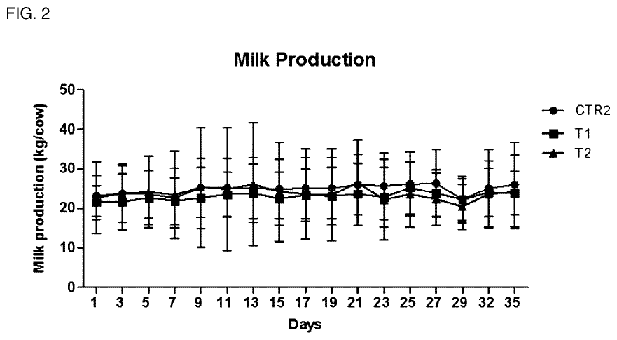

As shown in FIG. 2, no significant difference was observed between the CTR2 group and either the T1 group or T2 group. Milk production was slightly increased from day 5 to day 25. However, milk production decreased from day 27.

Fat, Protein, and Lactose in Milk. Somatic Cell Count (SCC).

The effects of OxBC on the amount of fat, protein, and lactose in the milk, and somatic cell count (SCC) are shown in Table 13. No significant difference (P>0.05) was observed over the three groups (CTR2, T1, and T2) except for protein content on day 28 and day 35 (P<0.05). Milk protein content of the T1 group was higher than the CTR2 group and T2 group. Although there was no statistically significant difference across the groups, there was a tendency for the SCC of the T1 group to be lower than the CTR2 and T2 groups.

TABLE 13
Effect of OxBC on Milk Content and SCC.

| Day | Content | CTR2 | T1 | T2 | SEM | P-value |
|---|---|---|---|---|---|---|
| 0 | Fat, % | 4.83 | 4.96 | 5.03 | 0.52 | 0.96 |
| 0 | Protein, % | 3.69 | 3.52 | 3.49 | 0.16 | 0.62 |
| 0 | Lactose, % | 4.51 | 4.81 | 4.66 | 0.13 | 0.26 |
| 0 | SCC, ×1000/mL | 3840 | 4050 | 4009 | 1152 | 0.99 |
| 7 | Fat, % | 3.84 | 3.68 | 3.32 | 0.44 | 0.66 |
| 7 | Protein, % | 3.81 | 3.64 | 3.44 | 0.14 | 0.17 |
| 7 | Lactose, % | 4.66 | 4.91 | 4.94 | 0.09 | 0.06 |
| 7 | SCC, ×1000/mL | 2607 | 1659 | 2461 | 1022 | 0.75 |
| 14 | Fat, % | 4.42 | 4.33 | 3.79 | 0.39 | 0.47 |
| 14 | Protein, % | 3.44 | 3.61 | 3.32 | 0.11 | 0.17 |
| 14 | Lactose, % | 4.84 | 4.96 | 4.96 | 0.12 | 0.69 |
| 14 | SCC, ×1000/mL | 1593 | 1079 | 1658 | 815 | 0.85 |
| 21 | Fat, % | 4.26 | 4.53 | 3.82 | 0.48 | 0.58 |
| 21 | Protein, % | 3.48 | 3.74 | 3.38 | 0.11 | 0.09 |
| 21 | Lactose, % | 4.82 | 4.99 | 4.86 | 0.15 | 0.71 |
| 21 | SCC, ×1000/mL | 1979 | 1285 | 2527 | 1044 | 0.70 |
| 28 | Fat, % | 4.06 | 3.79 | 3.21 | 0.32 | 0.13 |
| 28 | Protein, % | 3.58^{ab} | 3.90^{a} | 3.41^{b} | 0.12 | 0.02 |
| 28 | Lactose, % | 4.86 | 4.97 | 5.00 | 0.09 | 0.47 |
| 28 | SCC, ×1000/mL | 1277 | 787 | 1452 | 679 | 0.76 |
| 35 | Fat, % | 3.83 | 4.56 | 4.05 | 0.44 | 0.43 |
| 35 | Protein, % | 3.30^{b} | 3.82^{a} | 3.53^{ab} | 0.12 | 0.01 |
| 35 | Lactose, % | 4.86 | 5.00 | 4.78 | 0.16 | 0.56 |
| 35 | SCC, ×1000/mL | 1594 | 1173 | 2646 | 911 | 0.45 |

Means in the same row with different superscript differ (P < 0.05)

### Microbiology

The results regarding total bacterial count (TBC) are summarized in Table 14. The difference was not significant between groups (P>0.05) from day 0 to day 21. On day 28 and day 35, total bacterial count of the T1 and T2 groups were lower than the CTR2 group (P<0.05). The correlation between SCC and TBC was not significant (SCC=0.07×TBC+2052.72; r²=0.0011, P=0.8945, n=18).

TABLE 14
Effect of OxBC on Total Bacterial Count (TBC) of Milk.

| Day | CTR2 | T1 | T2 | SEM | P-value |
|---|---|---|---|---|---|
| 0 | 1142.61 | 242.98 | 396.00 | 375.62 | 0.18 |
| 7 | 383.72 | 319.10 | 1188.11 | 342.45 | 0.15 |
| 14 | 521.49 | 379.55 | 115.14 | 336.68 | 0.68 |
| 21 | 1685.56 | 1503.63 | 411.20 | 681.92 | 0.31 |
| 28 | 717.338 | 344.17b | 109.85b | 134.67 | 0.01 |
| 35 | 657.008 | 139.78b | 82.00b | 110.87 | <0.01 |

Means in the same row with different superscript differ (P < 0.05)

### Conclusion

The addition of OxBC did not affect DMI and milk production of the dairy cows (P>0.05), the fluctuations of the milk production may be related to weather and management factors. The results obtained from the present experiment demonstrated that cows fed a diet supplemented with 30 ppm of OxBC showed milk protein concentration higher than the CTR2 group, while the SCC of the T1 group decreased from the first week compared to the CTR2 and the T2 groups. Bovine mastitis is a result of inflammation of the mammary gland. At the moment, SCC remains the method of choice to monitor the udder status of the cows. The decrease of SCC of the T1 group reflected the therapeutic effect of 30 ppm of OxBC supply on mastitis.

In the current study, milk total bacterial count of the T1 and T2 groups were generally lower than the CTR2 group although the correlation between SCC and TBC was not significant (P=0.8945, n=18). Many factors could influence the TBC of milk. The degree of inflammation is dependent on the nature of the causative agent and on age, breed, immunological health and lactation state of the animal ((Viguier, et al., (2009) Detection: current trends and future perspectives. Trends in Biotechnology, 27, 486-493).

In conclusion, supplementation with 30 ppm OxBC provided meaningful improvement to milk protein content, and resulted in a decreased SCC of the milk, while supplementation with 60 ppm OxBC showed no significant effect on SCC. In addition, supplementation with 30 and 60 ppm OxBC both reduced TBC of the milk.

### Example 3

### Effect of Dietary OxBC on Treatment of Subclinical Mastitis in Herds of Lactating Dairy Cows

The following study was conducted to further investigate the ability of dietary OxBC supplementation in treating subclinical mastitis in dairy cattle.

### Negative Controlled Intervention Study

This negative controlled intervention study was conducted using cows sourced from 4 commercial dairy herds located in the North Island of New Zealand.

Cows were selected based on having an SCC>200,000 cells/mL at the most recent herd (DHIA) test and having no record of treatment with antimicrobials or nonsteroidal anti-inflammatories in the 14 days preceding sampling. The teat ends of cows were examined, and milk samples collected from each individual quarter for bacteriology and SCC determination.

Quarters with an SCC>200,000 cells/mL and from which bacteria were isolated were enrolled. Cows were blocked by age (2 years versus >2 years), ranked on herd test SCC, and then assigned randomly within sequential pairs of cows to be fed with a 0.5 kg of a pelletised cereal-based feed that did or did not include 3 g of OxBC premix. The OxBC used in this study was the commercial product, OxC-beta™ Livestock 10%. Herd owners assessed cows daily and those with grossly evident signs of clinical mastitis were sampled for microbiology culture and treated as per normal farm protocol. Enrolled quarters were resampled 21 and 42 days after commencement of treatment for bacteriology and SCC.

Incorporation of OxBC in the form of OxC-beta™ Livestock 10% in the diet of cows for 42 days increased the bacterial cure rate (e.g., to resolve subclinical mastitis and reduce risk of clinical mastitis) in comparison with control-fed cows. These effects are interpreted as being a consequence of improved immune function in the OxBC supplemented cows. Providing sufficient levels of immune-supporting beta-carotene copolymers through supplementing with OxBC helped the immune system to function at an optimal level in the OxBC groups. It is again noted that OxBC has been shown to possess no antimicrobial activity, therefore the reduction in the number of bacterial infections is not due to any direct antimicrobial activity of the product.

### Materials and Methods

### Start of Animal Phase

The selection and sampling of the animals occurred between 6 and 15 days after the herd test. Initiation of feeding occurred once microbiology and SCC results were available and cows had been assigned to treatment.

### Animals

Each cow was identified by an individual management number (“cow number”) that was unique within each herd. The identification was by plastic ear tags. Additionally, all study animals had lifetime identities and electronic identification (EID) tags. No acclimation was required. Cows were subjected to normal preventative health programs practiced in the herd. Table 15 shows the inclusion criteria for the cows in the study.

TABLE 15
Inclusion Criteria

| | |
|---|---|
| Species: | Dairy cows |
| Number: | 227 sampled in total |
| Breed: | Friesian (i.e. > 12/16th Friesian), Jersey (> 12/16th Jersey), |
| | or Cross bred (all others) |
| Age: | ≥2 years old |
| Gender: | Female |
| Weight: | Open (approx. 300-800 kg) |
| Source: | Commercial or research herds |
| Health status: | Healthy animals |
| Special | SCC >200,000 cells/mL at most recent herd test and not |
| requirements: | treated with antibiotics or NSAID (nonsteroidal anti- |
| | inflammatory drug) in preceding 14 days |

### Herds

Herds were enrolled on the basis of:
presence of an individual cow identification system and willingness to allow electronic access to cow records;
willingness to follow the experimental protocol;
availability of herd test (DHIA) data; and
willingness to record all animal health events (metabolic disorders, mastitis, lameness, retained placenta, and uterine infections) and the details of animal health treatments.

### Animal Environment, Feeding, and Management

The cows were managed under pastoral New Zealand dairy management systems, that is managed on pasture and not housed. Cows were fed predominantly on rye grass/white clover pasture swards. Supplementary feeding was at the discretion of herd owner or manager, but cows in both treatment groups were managed in such a way that they had equal access to any supplementary feed (see Table 17). Water was available ad libitum.

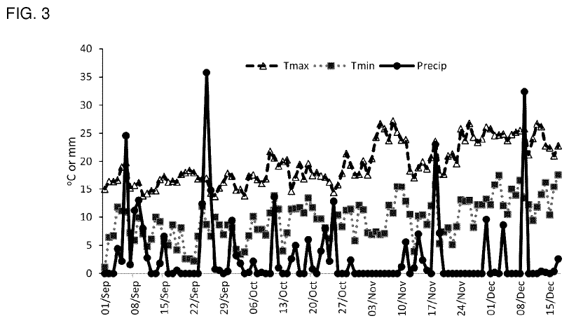

The daily maximum and minimum temperature and 24 hour precipitation from the Ruakura weather station (Lat −37.7739, Long 175.3052) for the study period are presented in FIG. 3. This data was obtained from NIWA (National Instituted of Water and Atmospheric Research).

Cows were subjected to the normal husbandry, health and management practices of the herd, with the exception that concomitant therapies were consistent with those identified above and below. Cows were managed in groups with cows not enrolled in the study. Cows enrolled in this study were milked consistent with normal practices at the study site, which was two times daily.

All cows in the study remained in the herds of origin at the end of the study.

### Procedures

Herd owners and staff were blinded to the treatment group to which animals were allocated. Technicians attended the farms daily to undertake the treatment feeding and the treatment feeds were colour-coded and were not labelled by treatment type. On-farm treatment sheets were similarly colour-coded. Hence neither the technicians providing the feed nor herd owners or their staff were aware of treatment allocations.

Samples were allocated a unique identifier such that laboratory staff (LIC, Cognosco) were unaware of the animal's identity or treatment group.

Responsibility for diagnosis of clinical mastitis was with the farm owner, manager and/or staff. As these individuals were blinded, no bias in clinical mastitis diagnosis should have occurred.

Within a herd, cows were blocked by age (2 years or >2 years), ranked by preceding herd test SCC, and then randomly assigned to the 2 treatment groups.

Cow data was obtained by downloading data from an electronic database (Mindapro, LIC, Newstead, Hamilton, NZ) and loaded into a purpose built database (Access, Microsoft, WA, USA). Following randomisation, lists of cows to be drafted for initial sampling were created and provided to the herd owner/manager.

Those cows were presented to trained research technicians and the teat end score assessed (Mein et al., Evaluation of bovine teat condition in commercial dairy herds: 1. Non-infectious factors. (2001)). Those animals with severe teat end damage (score=“very rough”) were excluded at this time point. Additionally, electronic and on-farm records of antimicrobial and NSAID treatments were assessed, and any cow treated in the preceding 14 days was excluded.

### Initial Sampling

Two milk samples (about 5 mL and about 25 mL) were collected from each quarter of each selected cow, following aseptic teat end preparation. These were held at 4° C. and processed for microbiology within 24 hours of collection and submitted for quarter-level SCC determination within 72 hours of collection.

Cows confirmed to have one or more quarters infected (i.e., presence of a recognised bacterial intramammary infection and a quarter-level SCC>200,000 cells/mL) were included in the study and randomised to treatment as outlined above.

### Treatment

Cows assigned to the OxBC group were fed about 0.5 kg of a cereal-based concentrate pellet containing OxBC daily for 42 days. The control group was fed about 0.5 kg of a cereal-based concentrate not containing OxBC daily for 42 days.

The cereal-based concentrate pellet was formulated and mixed by a commercial feed mill (Seales Winslow, Morrinsville, NZ). The pellet contained 25% wheat, 25% maize, 30% palm kernel expeller, 8% broil, 4% dried distillers' grain, 8% molasses, and 0.02% of a sweetener (rumasweet). The treatment diet included 0.6% of the OxC-beta™ Livestock 10% premix. Thus, treatment animals were fed approximately 3 g per cow per day of the OxC-beta™ Livestock 10% commercial product which corresponds to 0.3 g per cow per day of the OxBC active.

Feeding was undertaken by placing the appropriate feed type into individual-cow feed bins in the milking parlour. This was undertaken by research technicians at daily visits to each farm. The feed was delivered using a measuring jug attached to a pole, which allowed the technicians to stand behind the cows in the milking parlour and deliver the feed to feed troughs in front of the cows. To facilitate identification of study animals, cows were marked with “tail paint” of two different colours that corresponded with the feed type. The delivery of feed to each enrolled animal on each day of feeding was individually recorded. Additionally, visual assessment of whether feed had been consumed by each individual animal and where feed was not consumed were noted. The mass of feed delivered each day to each group within each herd was calculated by weighing the feed bags before and after each day's feeding, as a cross-check that the appropriate mass of feed had been delivered. The estimated mean (±standard deviation (SD)) daily intake of the feed is presented in Table 16.

TABLE 16
Average daily amount of control and OxBC containing
dairy pellets offered in kilograms/cow/day (as fed)

| | Control | | OxBC | |
|---|---|---|---|---|
| Herd | Mean | SD | Mean | SD |
| Farm A | 0.54 | 0.01 | 0.54 | 0.01 |
| Farm B | 0.53 | 0.04 | 0.52 | 0.04 |
| Farm C | 0.52 | 0.01 | 0.53 | 0.01 |
| Farm D | 0.52 | 0.02 | 0.51 | 0.02 |

### Post-Sampling Procedures and Observations

At Days 21 and 42 after initiation of treatment, quarter-level milk samples were collected from each enrolled quarter following aseptic teat end preparation for microbiology and SCC.

Microbiology was undertaken by trained technicians at AnexaFVC, Morrinsville. Microbiology was undertaken following the procedures recommended by the National Mastitis Council, USA. Briefly, 10 μL of milk was streaked onto a quarter of a 5% blood agar plate containing 0.1% esculin (Fort Richard, Auckland, New Zealand), and incubated at 37° C. for 48 hours.

The genus of bacteria was determined on the basis of colony morphology, Gram stain, and catalase reaction. Gram positive, catalase positive isolates were tested with the coagulase test to differentiate CNS from S. aureus. Gram positive, catalase negative were assessed by esculin reaction, CAMP test and growth in inulin and SF broth. Coliforms were sub-cultured on MacConkeys agar, triple iron sugar, citrate, and motility test were performed. Where the identity of the isolate was unclear using conventional biochemical tests, isolates were submitted for MALDI TOF testing. Samples were identified using a unique sample identification number generated from within the purpose built database.

In some cases, 2 distinct bacterial species were isolated from a milk sample. These were reported individually and used in the analysis of new intramammary infection rates. However, for reporting they are coded as mixed major infections (i.e., a major pathogen (e.g., Staphylococcus aureus, Streptococcus uberis, Streptococcus dysgalactiae)) when isolated in conjunction with a minor pathogen (e.g., coagulase-negative Staphylococcus (CNS) or Corynebacterium species) or as mixed minor infections where 2 different minor pathogens were present.

The SCC was determined using a fluoro-optic methodology (Foss, Hillerod, Denmark) at the laboratories of LIC (Riverlea, Hamilton). Results were forwarded to Cognosco as comma separated variable (CSV) files which were loaded into the purpose-built database

No adverse events occurred during the study. As expected, a number of animals were treated for clinical disease during the course of the study (e.g., mastitis and lameness) as outlined below. No animal exhibited systemic disease and hence no additional veterinary visit occurred to any enrolled animal during the course of the study.

### Concomitant Therapies While on Study

Products containing antibiotics or non-steroidal anti-inflammatory drugs (NSAID) were administered where animal welfare was believed to be at risk. The reason and type of all treatments was recorded. Each treatment was assessed for impact on the study outcomes.

No enrolled animals died or were euthanised during the study and hence no necropsies were undertaken.

Reasons for Cows Removed from Study

One cow was injured during the milking process on Day 35 post initiation of feeding. Due to this injury this cow was unable to be brought to the milking parlour between Days 35 and 42 of the study. Hence, this cow was not fed the appropriate feed over this time and was not sampled at Day 42. The bacteriological and SCC for the quarters of this cow could not be determined and the cow was excluded from the final analyses.

### Results

The experimental unit for this study was the quarter.

A total of 267 cows were selected for initial assessment and sampling based on having had an elevated SCC at the preceding herd test. Of these, 227 were milk sampled. The remaining 40 were not sampled as they had been culled, were not presented, were diagnosed with clinical mastitis at the time of initial assessment, or had very rough teat ends. Seventy-one cows had no quarters that met the quarter-level enrollment criteria (i.e., quarter level SCC>200,000 cells/mL and presence of bacteria upon culture) and were not enrolled. Thus, 156 cows had one or more quarters that met the enrollment criteria and following blocking for age were assigned to feed treatments.

There were 77 versus 79 cows and 135 versus 129 quarters that were assigned to the control and treatment feeding groups, respectively (Table 17). Three cows were diagnosed with clinical mastitis following initially sampling, but before commencement of feeding. These cows were excluded from the study prior to initiation of feeding.

Two cows were treated with systemic antimicrobials for lameness post enrollment (one in the treatment group and one from the control group, both with only one enrolled quarter). A total of 5 cows and 7 quarters were diagnosed with clinical mastitis following enrollment: 4 cows and 6 quarters from the control group; and 1 cow and 1 quarter from the treatment group.

TABLE 17
Fate of cows selected and enrolled in a study assessing the effect
of oral supplementation with OxBC on cure of subclinical mastitis

| | No. | No. quarters | |
|---|---|---|---|
| | cows | Control | OxBC |
| Selected for sample 1 | 267 | | |
| Not sampled | 40 | | |
| Sample 1 taken | 227 | | |
| Did not meet SCC/microbiology criteria | 71 | | |
| Assigned to feed group | 156 | | |
| Excluded before feeding started | 3 | | |
| Started feeding | 153 | | |
| Sampled D 21 | 153 | 135 | 129 |
| Sampled D 42 | 152 | 135 | 128 |
| Clinical mastitis post enrollment | 5 | 6 | 1 |
| Cow treated for any other reason post | 2 | 1 | 1 |
| enrollment | | | |

### Outcome Variables

The key outcome variable was bacterial cure rates. Bacterial cure rates were defined as having occurred where the bacteria present prior to initiation of treatment was not isolated from either the D 21 or D 42 samples. Bacterial cure was defined as occurring even where a different bacterial species were identified at the post treatment sampling. Enrolled quarters that were diagnosed with clinical mastitis within 42 days of commencement of feeding were defined as bacterial cure rate failures. Where a milk sample from a quarter that was defined as contaminated (n=3) or where a sample was not collected as the animal was not presented (n=1), the status of the quarter post-treatment could not be defined and hence bacterial cure was coded as a null.

The quarter- and cow-level SCC, milk yield, and clinical mastitis incidence rate were also analysed.

### Statistical Design and Analysis

The balance of animals assigned to the 2 treatment groups was assessed using chi-squared analysis for categorical variables including herd, age group (categorised as heifer (primiparous) versus cow (multiparous)), breed (categorised as ≥ 12/16ths Friesian as Friesian, otherwise crossbred or Jersey), and days in milk at commencement of feeding (categorised as 15-60, 61-75, 76-90, ≥91 days)); and one-way analysis of variance for the natural log of the cow-level SCC at the herd test preceding commencement of feeding.

Multivariable models were used to estimate the effect of treatment (that is, OxBC vs. no OxBC supplementation (control). Potential confounding variables including age (i.e. primiparous vs multiparous), breed (Friesian vs other), preceding quarter-level SCC, quarter location (i.e. rear vs fore glands), days in milk (DIM) at commencement of feeding, and intramammary infection type (categorised as major vs minor) were considered during the modelling process. Initially bivariate analysis was undertaken with each of the potential explanatory variables tested for bacterial cure rates using chi-squared analysis (for categorical variables) or logistic regression (for continuous variables). Those variables associated (P<0.2) were then used in a manual forward step wise model building process. Variables remained in the final models where they were significant (i.e. P<0.05) or removal resulted in more than 20% change in the coefficient for the effect of treatment group. For bacterial cure rates, the final model was a mixed effect binary logistic regression models which included cow as a random effect. Attempts to create a 3 level model (i.e. quarter within cow within herd) resulted in a failure of the model to converge, due to the relatively small number of cows with multiple quarters enrolled. Note as no primiparous animals met the enrollment criteria in one herd (Speldhurst), models that included age also failed to converge. Thus, this variable was removed from modelling. For the initial logistic regression models model fit was assessed using the Hosmer Lemeshow test, the link test and by assessing change in the BIC when including/excluding variables. Data are reported as the odds ratio (OR) of the treatment effect as well as the estimated marginal mean and 95% confidence intervals.

Quarter level SCC data was natural log (ln) transformed prior to multilevel repeated-measures generalised linear regression modelling with gland nested within cow nested within herd. Fixed effects included treatment group and day (i.e. D 0, 21, 42) relative to start of feeding. The interaction of Treatment by Day was forced into the model. The estimated marginal mean was derived from the final model and pairwise comparison of marginal means by treatment group and day undertaken using pairwise comparisons and using the Bonferroni correction.

The incidence of clinical mastitis diagnosis within 42 days of commencement of feeding was analysed using logistic regression with the standard error calculated clustered by herd.

The quarter level SCC was also recoded as being <200,000 cells/mL, or ≥200,000 cells/mL, and this categorical variable then analysed using a multilevel, repeated measures logistic regression model with gland nested within cow nested within herd. Fixed effects included treatment group and day (i.e. D 21, 42) relative to start of feeding. Note by design only quarters with an SCC>200,000 cells/were included in the study, hence all quarters by definition had an SCC greater than this on Day 0. The interaction of Treatment by Day was forced into the model. The estimated marginal mean was derived from the final model and pairwise comparison of marginal means by Treatment group and Day undertaken using the Bonferroni correction.

The cow composite (herd test) SCC, milk yield (kg/cow/d) and milk solids (i.e. sum of kg of milk fat and milk protein/cow/day) from immediately prior to initiation of treatment and the next herd test during or just after the period of treatment were analysed using multilevel repeated-measures generalised linear regression modelling with cow nested within herd. SCC was natural log transformed for analysis. Fixed effects included Treatment and whether the test was pre-or post-initiation of the start of feeding. The interaction of Treatment by time was forced into the model. Other explanatory variables (e.g. primiparous vs multiparous; breed coded as Friesian vs others; DIM at herd test) were offered to the model and included where significant (P<0.05) and/or resulted in changes in the coefficient for Treatment of >20%. The estimated marginal mean was derived from the final model and pairwise comparison of marginal means by treatment group and time undertaken using the Bonferroni correction. Post initiation of treatment herd tests occurred at 36, 55, 31, and 57 days after initiation of feeding for Farms B, D, A, and C herds, respectively.

Analyses were undertaken in STATA v16 (STATA Corp, College Station Tex., USA).

### Herd Data

Descriptive data about enrolled herds including herd size, dry matter intake and calcium and magnesium intakes are presented in Table 18.

TABLE 18
Descriptive data and feed intake for enrolled herds.

| Farm Name | Farm A | Farm B | Farm C | Farm D |
|---|---|---|---|---|
| No. lactating cows | 799 | 424 | 777 | 632 |
| Approx, intake | | | | |
| (kg DM/cow/day) of: | | | | |
| Pasture | 14 | 15 | 14 | 15.5 |
| Palm Kernel Expeller | | 4 | | |
| Maize silage | 2 | 1 | | |
| Dried distillers’ grain | | 0.6 | | |
| Corn gluten | | 0.6 | | |
| Dairy meal | | | 2 | 2 |
| Tapioca | 1 | | | |
| Total | 17 | 21.2 | 16 | 17.5 |
| Intake (g/cow/day) of: | | | | |
| Magnesium (as MgO) | 29 | 57 | 23 | 15 |
| Calcium (as CaCO3) | 29 | 40 | 40 | 60 |
| Dairy NZ farm system | 3 | 5 | 3 | 2 |
| type (1-5) | | | | |

### Group Balance

There was no difference between the treatment groups in terms of distribution within herd, breed, age group, or DIM at the time of commencement of feeding (all P>0.8; Table 19). There was no difference in the natural log of the herd test SCC preceding initiation of feeding (6.18 (SD=0.77) versus 6.18 (SD=0.72) for log herd test SCC for the control versus OxBC treatment groups, respectively; P=0.96).

Table 19 shows the number of cows assigned to be fed the control diet or the diet containing the OxBC by herd, breed, age, and days in milk (DIM) on the first day of feeding. The P-values are from chi-squared analyses. Note animals assigned to feeding treatment but that did not actually get fed are excluded.

TABLE 19
Number of cows assigned to be fed control diet or
diet containing OxBC by herd

| Variable | level | Control | OxBC | Total | P-value |
|---|---|---|---|---|---|
| Herd | Farm B | 7 | 7 | 14 | 0.98 |
| | Farm D | 13 | 15 | 28 | |
| | Farm A | 34 | 34 | 68 | |
| | Farm C | 22 | 21 | 43 | |
| Breed | Friesian | 29 | 29 | 58 | 0.95 |
| | Other | 47 | 48 | 90 | |
| Age | Heifer | 7 | 7 | 14 | 0.98 |
| | Cow | 69 | 70 | 139 | |
| DIM at feed | 15-60 d | 20 | 24 | 44 | 0.81 |
| | 61-75 d | 18 | 19 | 37 | |
| | 76-90 | 26 | 21 | 47 | |
| | 91 + d | 12 | 13 | 25 | |

### Descriptive Microbiology

The minor pathogens (i.e. CNS and Corynebacterium species) were the most common isolates both prior to commencement of feeding (Day 0) and at D 21 and D 42 post initiation of feeding (Table 21).

Table 20 shows bacterial diagnosis of quarters from cows assigned to supplementary feed with OxBC (control) or containing added OxBC by day relative to commencement of feeding. D 0 is the quarter level sample results prior to initiation of feeding, while D 21 and 42 are the samples collected post initiation of feeding.

TABLE 20
Bacterial diagnosis

| | D 0 | | | D 21 | | | D 42 | | |
|---|---|---|---|---|---|---|---|---|---|
| Bacteria | Control | OxBC | Total | Control | OxBC | Total | Control | OxBC | Total |
| No growth | 0 | 0 | 0 | 21 | 19 | 40 | 15 | 24 | 39 |
| S. aureus | 11 | 10 | 21 | 9 | 8 | 17 | 11 | 11 | 22 |
| S. dysgalactiae | 1 | 1 | 2 | 4 | 1 | 5 | 3 | 2 | 5 |
| S. uberis | 14 | 20 | 34 | 8 | 17 | 25 | 11 | 15 | 26 |
| Mixed major¹ | 3 | 2 | 5 | 1 | 1 | 2 | 3 | 4 | 7 |
| CNS | 34 | 30 | 64 | 25 | 25 | 50 | 26 | 20 | 46 |
| Corynebacterium spp. | 66 | 52 | 118 | 59 | 43 | 102 | 53 | 45 | 98 |
| Mixed minor² | 1 | 2 | 3 | 3 | 9 | 12 | 8 | 2 | 10 |
| Serratia spp. | 0 | 1 | 1 | | | | | | |
| Yeasts | 5 | 11 | 16 | 4 | 4 | 8 | 4 | 5 | 9 |
| Total | 135 | 129 | 264 | 135 | 129 | 264 | 135 | 128 | 263 |

¹Mixed major infections are milk samples from which both a major pathogen (i.e. S. aureus, S. dysgalactiae or S. uberis) and a minor pathogen (i.e. CNS or Corynebacterium species) were isolated
²Mixed minor infections are milk samples from which two minor pathogen (i.e. CNS or Corynebacterium species) were isolated

### Bacterial Cure Rates

Initial bivariate model found an association between bacterial cure rates and Treatment, age, DIM, front versus rear gland, herd, and quarter level SCC. Breed and bacteriological species pre-treatment were not associated at the bivariate level with bacterial cure rate.

In the final model, only Treatment group remained in the model. More quarters from cows fed OxBC underwent cure of bacterial infection compared with quarters from cows fed the control feed (13.9 (95% CI 4.1-23.7) % vs 6.9 (95% CI 4.8-9.1) % for quarters from OxBC fed vs control fed cows, respectively; P=0.02; odds ratio=2.18 (95% CI 1.14-4.17)).

### Quarter Level SCC

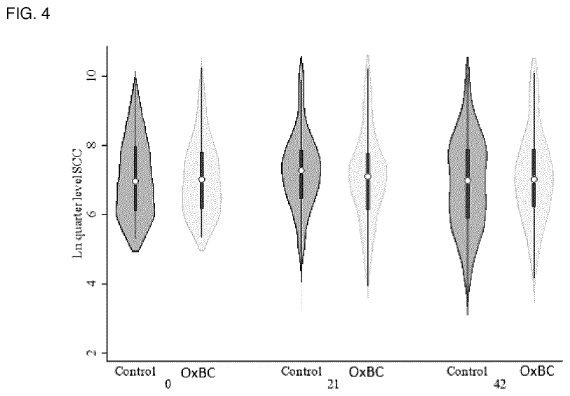

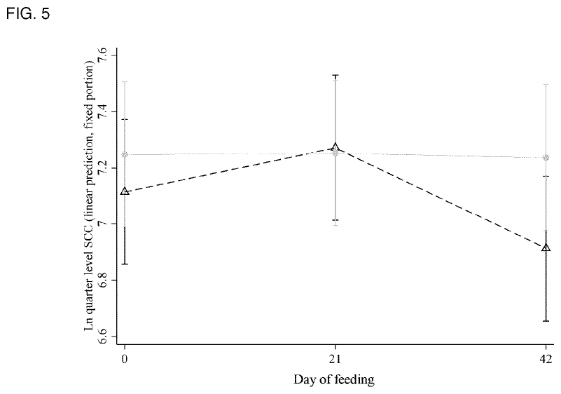

There was no effect of Treatment (P=0.34) or Day (P=0.12), nor was there a Treatment by Day interaction (P=0.17) for the quarter level ln SCC (FIG. 4 and FIG. 5).

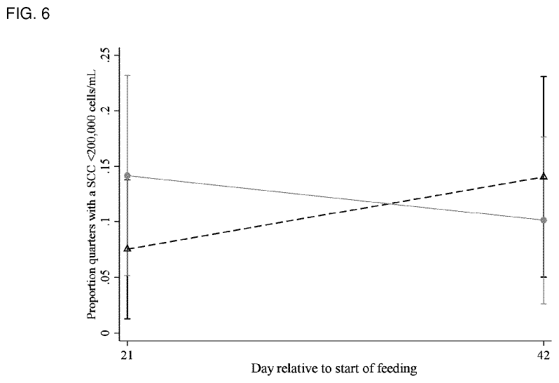

There was no effect of Treatment (P=0.56) or Day (0.64) on the proportion of quarters with an SCC<200,000 cells/mL. However, there was a Treatment by Day interaction (P=0.05; FIG. 6). There was a tendency (P=0.07) for more resolved quarters to have an SCC<200,000 cells/mL relative to unresolved quarters ( 9/52 (17.3%) vs 43/463 (9.3%).

### Clinical Mastitis Incidence

Fewer of the quarters in cows fed OxBC had clinical mastitis in the 42 days post initiation of feeding compared with quarters from control fed cows [ 1/129 (0.78 (exact binomial 95% CI 0.02-4.24)%)] vs 6/135 [4.44 (exact binomial 95% CI 1.65-9.42)%)]. The odds ratio of mastitis diagnosis in quarters from cows fed OxBC was 0.17 (95% CI 0.03-0.82) relative to quarters from cows fed the control diet. The one quarter of the one cow in the treatment group which was diagnosed with mastitis was diagnosed 41 days after commencement of feeding. This cow was chronically infected with Staphylococcus aureus.

### Cow-Level Milk-Quality and Yield

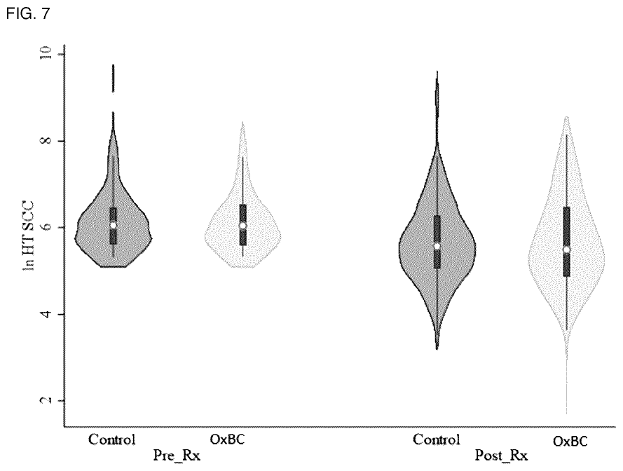

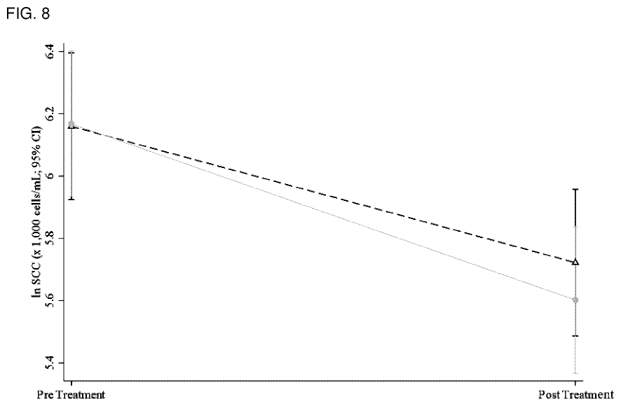

The in cow-level (herd test) SCC declined following initiation of feeding (P<0.001), but there was no effect of Treatment (P=0.66), nor was there a Treatment by Time interaction (P=0.56; FIG. 7 and FIG. 8).

Milk yield was unaffected by OxBC feeding (22.9 (95% CI 21.0-24.8) vs 23.1 (95% CI 21.2-25.1) kg/cow/day for cows in the OxBC vs control fed group, respectively; P=0.71). Milk yield was lower at the post-treatment production recording (21.0 (95% CI 19.0-22.9) vs 25.0 (95% CI 23.1-27.0) kg/cow/day at the post treatment vs pre-treatment herd test, respectively; P<0.001). There was no Treatment by Time interaction (P=0.84; FIG. 6). Friesians produced more milk than crossbreds or Jersey cows (24.4 (95% CI 22.4-26.4) vs 22.3 (95% CI 20.3-24.2) kg/cow/day for Friesian's vs other breeds, respectively; P=0.005), and primiparous animals produced less milk than multiparous animals (19.2 (95% CI 16.5-21.8) vs 23.4 (95% CI 21.6-25.3) kg/cow/day for primiparous vs multiparous cows, respectively; P<0.001).

Milk fat percentage was unaffected by OxBC feeding [4.72 (95% CI 4.49-4.96) % vs 4.54 (95% CI 4.41-4.89%) for cows in the OxBC vs control fed group, respectively; P=0.41; FIG. 7a]. Milk fat percentage was lower at the post-treatment production recording [4.60 (95% 014.36-4.82) % vs 4.78 (95% CI 4.54-5.01) % at the post treatment vs pre-treatment herd test, respectively; P=0.04]. There was no Treatment by Time interaction (P=0.68; FIG. 7b). Friesians had a lower milk fat percentage than crossbreds or Jersey cows [4.55 (95% CI 4.30-4.80) % vs 4.76 (95% CI 4.53-5.00) % for Friesian's vs other breeds, respectively; P=0.05].

Milk protein percentage was unaffected by OxBC feeding [3.71 (95% CI 3.64-3.78) % versus 3.71 (95% CI 3.65-3.78) % for cows in the OxBC vs control fed group, respectively; P=0.92; FIG. 8a]. Milk protein percentage was lower at the post-treatment production recording [3.66 (95% 013.59-3.72) % vs 3.77 (95% CI 3.70-3.83) % at the post treatment vs pre-treatment herd test, respectively; P=0.003]. There was no Treatment by Time interaction (P=0.83; FIG. 8b) and no effect of breed (P=0.80).

### Discussion

This controlled randomised intervention study demonstrated that oral supplementation of lactating dairy cows with subclinical intramammary infections with OxBC resulted in an increased cure rate of bacterial infection and a reduced risk of subsequent clinical mastitis, relative to control fed cows. OxBC supplementation had no effect on either quarter-, or cow-level SCC or on milk yield.

A total of 135 and 129 quarters were allocated to the treatment (OxBC) and control group, respectively. The bacterial cure rate was only 7% in the control group, and 14% in the treatment group. More stringent enrollment criteria in the current study may have resulted in more significant subclinical infections being selected for study and that may have produced a lower bacterial cure rate in the control group.

Resolution of bacterial infection was associated with a lower quarter level SCC. However, despite a higher resolution rate in the quarters from cows fed OxBC, there was no difference in quarter or cow level SCC between feed treatment groups. It is also interesting to note that less than 20% of resolved quarters had SCC<200,000 cells/ml by Day 42, illustrating the long period of inflammation that occurs in the mammary gland despite apparent removal of the bacterial pathogens.

It is concluded that oral supplementation of lactating dairy cows with OxBC results in a higher bacterial cure rate and lower incidence of subsequent clinical mastitis in quarters with subclinical mastitis than for quarters from cows fed the control diet without the additional OxBC. There was no effect of OxBC on milk yield or composition.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A method for treating subclinical mastitis in a mammal, said method comprising feeding said mammal an animal feed comprising an effective amount of carotenoid-oxygen copolymer.

2. The method of claim 1, wherein the mammal is lactating and/or nursing offspring.

3. The method of claim 1 or 2, wherein the mammal is cattle, horses, dogs, cats, sheep, or swine.

4. The method of claim 3, wherein the mammal is dairy cattle.

5. The method of claim 4, wherein the dairy cattle is selected from Holstein cattle, Jersey cattle, Brown Swiss cattle, Guernsey cattle, Ayrshire cattle, Milking Shorthorn cattle, and Red and White Holstein.

6. The method of claim 3, wherein the mammal is swine.

7. The method of any one of claims 1 to 6, wherein the feeding is from 10 days to 50 days prior to the collection of milk from the mammal.

8. The method of any one of claims 1 to 7, wherein the feeding is ongoing during the collection of milk from the mammal.

9. The method of any one of claims 1 to 8, wherein the method comprises ameliorating one or more symptoms of subclinical mastitis.

10. The method of any one of claims 1 to 8, wherein the method comprises reducing the frequency of subclinical mastitis progressing to full clinical mastitis in the mammal.

11. A method of improving the health of offspring of a mammal, said method comprising feeding said mammal an animal feed comprising an effective amount of carotenoid-oxygen copolymer after impregnation of the mammal.

12. The method of claim 11, wherein the mammal is selected from cattle, horses, dogs, cats, sheep, and swine.

13. The method of claim 12, wherein the mammal is swine.

14. The method of any one of claims 11 to 13, wherein the feeding is begun from 10 days to 30 days prior to the impregnation of the mammal.

15. The method of any one of claims 11 to 13, wherein the feeding is ongoing throughout the gestation period of the offspring.

16. The method of claim 14 or 15, further comprising continuing feeding the mammal during the period in which the mammal nurses the offspring.

17. The method of any one of claims 11 to 16, wherein the transfer of passive immunity from the pregnant mammal to the offspring is enhanced.

18. The method of any one of claims 11 to 16, wherein the mammal has or is at risk of subclinical mastitis prior to or subsequent to the impregnation.

19. A method for reducing physiological stress in a lactating mammal, said method comprising feeding said mammal an animal feed comprising an effective amount of carotenoid-oxygen copolymer during a period in which the mammal is lactating.

20. The method of claim 19, wherein the mammal is nursing offspring.

21. The method of claim 19 or 20, wherein the mammal is cattle, horses, dogs, cats, sheep, or swine.

22. The method of claim 21, wherein the mammal is dairy cattle.

23. The method of claim 22, wherein the dairy cattle is selected from Holstein cattle, Jersey cattle, Brown Swiss cattle, Guernsey cattle, Ayrshire cattle, Milking Shorthorn cattle, and Red and White Holstein.

24. The method of claim 21, wherein the mammal is swine.

25. The method of any one of claims 19 to 24, wherein reducing physiological stress comprises reducing fat loss in the mammal.

26. The method of any one of claims 19 to 24, wherein the mammal has or is at risk of subclinical mastitis during the lactation.

27. A method for improving reproductive performance of a mammal following the birth of offspring by the mammal, said method comprising feeding said mammal an animal feed comprising an effective amount of carotenoid-oxygen copolymer.

28. The method of claim 27, wherein the mammal is selected from cattle, horses, dogs, cats, sheep, or swine.

29. The method of claim 28, wherein the mammal is dairy cattle.

30. The method of claim 29, wherein the dairy cattle is selected from Holstein cattle, Jersey cattle, Brown Swiss cattle, Guernsey cattle, Ayrshire cattle, Milking Shorthorn cattle, and Red and White Holstein.

31. The method of claim 28, wherein the mammal is swine.

32. The method of any one of claims 27 to 31, wherein improving reproductive performance is reducing the number of days required for the mammal to return to estrus.

33. The method of any one of claims 27 to 32, wherein the mammal has or is at risk of subclinical mastitis prior to or subsequent to impregnation of the mammal.

34. A method for reducing bacteria count in colostrum or milk of a mammal, said method comprising feeding said mammal an animal feed comprising an effective amount of carotenoid-oxygen copolymer.

35. The method of claim 34, wherein the mammal is lactating and/or nursing offspring.

36. The method of claim 34 or 35, wherein the mammal is cattle, horses, dogs, cats, sheep, or swine.

37. The method of claim 36, wherein the mammal is dairy cattle.

38. The method of claim 37, wherein the dairy cattle is selected from Holstein cattle, Jersey cattle, Brown Swiss cattle, Guernsey cattle, Ayrshire cattle, Milking Shorthorn cattle, and Red and White Holstein.

39. The method of claim 36, wherein the mammal is swine.

40. The method of any one of claims 34 to 39, wherein the feeding is from 10 days to 50 days prior to the collection of milk from the mammal.

41. The method of any one of claims 34 to 40, wherein the feeding is ongoing during the collection of milk from the mammal.

42. The method of claim 40 or 41, wherein the shelf-life of the milk is increased.

43. The method of any one of claims 34 to 42, wherein the mammal has or is at risk of subclinical mastitis.

44. The method of any one of claims 1 to 43, wherein the animal feed comprises from 0.0001% to 0.005% (w/w) carotenoid-oxygen copolymer.

45. A method for producing pasteurized milk, said method comprising:
(i) providing milk obtained from a mammal, wherein mammal was fed an animal feed comprising an effective amount of carotenoid-oxygen copolymer during a period beginning at least from 10 days to 30 days prior to the collection of milk from the mammal; and
(ii) processing the milk using a low temperature pasteurization process to produce said pasteurized milk.

46. The method of claim 45, wherein the mammal was fed an animal feed comprising an effective amount of carotenoid-oxygen copolymer during a period beginning at least from 30 days to 50 days prior to the collection of milk from the mammal.

47. The method of claim 46, wherein the mammal was fed an animal feed comprising an effective amount of carotenoid-oxygen copolymer during a period beginning at least from 35 days to 45 days prior to the collection of milk from the mammal.

48. The method of claim 45, wherein the mammal was fed an animal feed comprising an effective amount of carotenoid-oxygen copolymer during a period beginning at least from 21 days to 42 days prior to the collection of milk from the mammal.

49. The method of any one of claims 45 to 48, wherein the shelf-life of the milk is increased.

50. The method of any one of claims 45 to 48, wherein the bacteria count in the milk is reduced.
